(19) United States
(12) Patent Application Publication
(10) Pub. No.: US 2016/0030349 A1
(43) Pub. Date: Feb. 4, 2016

(54) NANOPARTICLES, METHODS OF PREPARATION, AND USES THEREOF

(71) Applicant: Boehringer Ingelheim Vetmedica GmbH, Ingelheim am Rhein (DE)

(72) Inventors: Donny FRANCIS, Frankfurt am Main (DE); Alf LAMPRECHT, Wesseling (DE); Martin FOLGER, Ingelheim am Rhein (DE); Ragna HOFFMANN, Biberach an der Riss (DE); Alfonso MARTIN-FONTECHA, Hannover (DE)

(21) Appl. No.: 14/810,921

(22) Filed: Jul. 28, 2015

(30) Foreign Application Priority Data
Aug. 1, 2014 (EP) 14179537.7

(51) Int. Cl. A61K 9/16; A61K 39/08

(52) U.S. Cl. CPC A61K 9/1676; A61K 39/08; A61K 9/1647

(57) ABSTRACT

The present invention relates to core-shell nanoparticles, methods for their production, and their use, in particular as adjuvants. Generally, the nanoparticles of the invention comprise a solid core consisting of a biodegradable polymer and a shell of amphiphilic molecules disposed about said core.

### BACKGROUND OF THE INVENTION

1. Technical Field

The present invention relates to core-shell nanoparticles, methods for their production, and their use, in particular as adjuvants. Generally, the nanoparticles of the invention comprise a solid core consisting of a biodegradable polymer and a shell of amphiphilic molecules disposed about said core.

2. Background of the Invention

Many vaccines are not immunogenic enough to elicit an immune response that would trigger immunity. Non-live-vaccines, for example, show a low immunogenic potency when administered alone. Moreover, proteins posing as antigens have to withstand harsh conditions to maintain their composition and thereby maintain their immunogenic potential. Recombinant proteins are safer to use than live, attenuated vaccines, but less immunogenic. Hence, substances that enhance the immune response of the safe, but poorly immunogenic antigens are in demand.

Adjuvants are compounds that increase and/or modulate the immune response, when used in combination with a specific antigen. The efficacy of many vaccines is dependent on the adjuvants as antigens have become more purified.

The application of an adjuvant can have different benefits and different adjuvant types are available. Immunomodulatory adjuvants can induce either a predominantly Th1 or Th2 type immune response, dependent on which adjuvants are being used. Another type of adjuvants are substances that can prolong the interaction between the antigen and antigen presenting cells. Furthermore, adjuvants have the potential to be antigen delivery systems that target antigen presenting cells like dendritic cells.

The exact mode of action of adjuvants is still not fully understood, but is has been suggested that, for some of them, a “depot effect” and an induction of an inflammation might be mechanisms of adjuvant effectiveness.

Many different adjuvants are available or currently in development. The most commonly used adjuvants are aluminum salts. They are FDA approved and safe to use in humans and animals. Generally, an aluminum hydroxide or aluminum phosphate gel is used, which binds the immunogenic substance via electrostatic interaction. A prolonged interaction of the antigen with cells of the immune system is possible because of the gel-like structure. Furthermore, it is suggested that aluminum salts activate the innate immune response, which in combination with the immunogenic substance subsequently leads to an adaptive immunity.

Freund's Adjuvant is an oil based adjuvant. It has been successfully used in veterinary vaccines, but remains inapplicable for humans, because of toxicity concerns. Freund's Adjuvant does however elicit a strong immune response, which can also be contributed to the high inflammatory effect of the mineral oil after administration.

A better approach than oil based adjuvants is an o/w-emulsion. MF59® and AS03 are examples for o/w-emulsions and are currently used in influenza vaccines. MF59® and AS03 induce a high degree of cell recruitment of monocytes and dendritic cells, which might be responsible for the adjuvant effects.

ISCOMs are matrices that are formed after interaction of saponins, cholesterol and phospholipids. These open cage-like structures have the immunogenic substance incorporated inside the cage. The mode of action is probably via targeting of immune cells. Pathogen-associated molecular patterns (PAMP) are viral and bacterial molecules that can be detected by PAMP receptors expressed by the host immune system. Toll-like receptors (TLR) are examples of PAMP receptors and can be found both in the surface (TLR-4) and in the cytoplasm (TLR-7/9) of animals and plants.

Adjuvants that pose as PAMPs (i.e. adjuvants that contain PAMP receptor agonists or adjuvants that activate PAMP receptors, respectively) can be ligands to toll-like receptors and therefore initiate an immune response. MPL as well as CpG have been identified as toll-like receptor agonists.

Adjuvants play an important role in vaccines and the battle against many diseases. However, only very few adjuvants have been approved for the use in humans and in animals.

Thus, novel, safe and efficient adjuvants are needed in view of the challenges of new, poorly immunogenic antigens, and as alternatives to overcome the limitations, in particular the side reactions, of the traditionally used adjuvants.

### DESCRIPTION OF THE INVENTION

The solution to the above technical problem is achieved by the description and the embodiments characterized in the claims.

Thus, the invention in its different aspects is implemented according to the claims.

The invention is based on the surprising finding that nanoparticles comprising a solid core of a biodegradable polymer which is coated with a shell of amphiphilic molecules may serve as safe and efficient adjuvants.

In one aspect, the invention thus relates to an amphiphile coated nanoparticle, wherein said nanoparticle is composed of: a solid core consisting of a biodegradable polymer, wherein optionally solvent molecules are included in the interior of the solid core; an amphiphile shell disposed over said solid core; and optionally, one or more antigens attached to said amphiphile and/or said solid core.

Said amphiphile coated nanoparticle, which is also termed the “nanoparticle of the present invention” hereinafter, has preferably a diameter lower than 250 nm or, more preferably, a size within a range of from 50 to 200 nm.

The biodegradable polymer described herein is in particular a synthetic polymer, wherein said synthetic polymer is preferably selected from the group consisting of polylactides, polyglycolides, polylactic polyglycolic copolymers, polyesters, polyethers, polyanhydrides, polyalkylcyanoacrylates, polyacrylamides, poly(orthoters), polyphosphazenes, polyamino acids, and biodegradable polyurethanes, and wherein a biodegradable polymer selected from the group consisting of poly(lactic-co-glycolic acid) (PLGA), Poly(Lactide-co-Glycolide) (PGA), Poly(lactic acid) (PLA), poly(ε-Caprolactone) PCL, Poly(methyl vinyl ether-co-maleic anhydride), PEG-PCL-PEG, and Polyorthoesters is particularly preferred.

The solvent molecules, as mentioned herein, are preferably molecules of a solvent selected from the group consisting of water, an organic solvent, and a combination thereof.

The term “amphiphile”, as used herein, refers to a chemical compound that includes a hydrophilic segment and a hydrophobic segment. In particular, the term “amphiphile” as used herein in the methods and compositions of the invention includes any agents that are capable of forming a structured phase in the presence of an aqueous solvent. Amphiphiles will have at least one polar, hydrophilic group and at least one non-polar, hydrophobic group. In particular, it is understood that the terms “amphiphile”, “amphiphilic molecule”, and “amphiphilic compound”, as used herein, are equivalent.

Preferably, the amphiphile is a surfactant or a PAMP receptor agonist, wherein the PAMP receptor agonist is in particular a Toll like receptor (TLR) agonist.

According to one preferred aspect, the amphiphile is a surfactant selected from the group consisting of an non-ionic, an anionic, and a cationic surfactant, and wherein said amphiphile is preferably: a non-ionic surfactant selected from the group consisting of: polyoxyethylene sorbitan fatty acid esters, sorbitan fatty acid esters, fatty alcohols, alkyl aryl polyether sulfonates, and dioctyl ester of sodium sulfonsuccinic acid; an anionic surfactant selected from the group consisting of sodium dodecyl sulfate, sodium and potassium salts of fatty acids, polyoxyl stearate, polyyoxylethylene lauryl ether, sorbitan sesquioleate, triethanolamine, fatty acids, and glycerol esters of fatty acids; or a cationic surfactant selected from the group consisting of didodecyldimethyl ammonium bromide, cetyl trimethyl ammonium bromide, benzalkonium chloride, hexadecyl trimethyl ammonium chloride, dimethyidodecylaminopropane, and N-cetyl-N-ethyl morpholinium ethosulfate.

More preferably, said amphiphile is a surfactant selected from group consisting of Polyvinyl alcohol (PVA), Polysorbate 20 (TWEEN® 20), Sodium dodecyl sulfate (SDS), Sodium cholate, and Cetyltrimethylammonium bromide (CTAB).

According to another preferred aspect, the amphiphile is selected from the group consisting of TLR (Toll like receptor) agonists, and wherein said amphiphile is preferably selected from the group consisting of a TLR1 agonist, a TLR2 agonist, and a TLR4 agonist.

More preferably, said amphiphile is a TLR agonist selected from the group consisting of: lipopolysaccharide (LPS) or a derivative thereof, lipoteichoic acid (LTA), Pam(3)CysSK(4) ((S)-[2,3-5w(palmitoyloxy)-(2-RS)-propyl]-N-palmitoyl-(R)-Cys-(S)-Ser-(S)-Lys4-OH or Pam3-Cys-Ser-(Lys)), Pam3Cys (S-[2,3-bis(palmitoyloxy)-(2RS)-propyl]-N-palmitoyl-(R)-cysteine or tripalmitoyl-S-glyceryl cysteine), Cadi-05, ODN 1585, zymosan, synthetic triacylated and diacylated lipopeptides, MALP-2, tripalmitoylated lipopeptides, a compound having a 2-aminopyridine fused to a five membered nitrogen-containing heterocyclic ring, Polyriboinosinic-polyribocytidylic acid (poly IC), a CpG oligodeoxynucleotides (ODNs), monophosphoryl lipid A (“MPL”), an imidazoquinoline compound (e.g. an amide substituted imidazoquinoline amine), a benzimidazole derivative, a C8-substituted guanine ribonucleotide, an N7, C8-substituted guanine ribonucleotide, bacteria heat shock protein-60 (Hsp60), peptidoglycans, flagellins, mannuronic acid polymers, flavolipins, teichuronic acids, ssRNA (single stranded RNA), dsRNA (double stranded RNA), or a combination thereof.

Still more preferably, said amphiphile is a TLR agonist selected from the group consisting of LPS, LPS derivative, and LTA.

Within the context of the invention, it is in particular understood that the term “derivative of LPS” is equivalent to the term “LPS derivative”. Thus, for instance, the term “lipopolysaccharide (LPS) or derivative thereof” is in particular equivalent to the term “lipopolysaccharide (LPS) or derivative of lipopolysaccharide (LPS)”.

As described herein, it is in particular understood that the term “amphiphile coated nanoparticle” is equivalent to the term “nanoparticle coated with (an) amphiphile” or with the term “nanoparticle coated with amphiphilic molecules”, respectively. Preferably, the amphiphile coated nanoparticle is a nanoparticle coated with a layer, preferably a monolayer, composed of molecules of an amphiphile.

The term “shell disposed over said core” as used herein is meant to refer to a coating layer, in particular a monolayer that surrounds the solid core of the nanoparticle. The term “amphiphile shell” as described herein in particular refers to a shell composed of amphiphilic molecules and is in particular equivalent to the term “shell composed of (an) amphiphile”. Preferably, the amphiphile shell is a layer, in particular a monolayer, composed of molecules of an amphiphile.

Preferably, the shell disposed over said solid core is a shell covering said solid core and being formed by a monolayer, preferably a closed monolayer, of a plurality of amphiphilic molecules, and wherein said monolayer is preferably composed of a plurality of molecules of an amphiphile.

The one or more antigens, as described herein, is (or are) in particular selected from the group consisting of PAMP receptor agonists, wherein the PAMP receptor agonists are preferably TLR (Toll like receptor) agonists, and wherein said one or more antigens is (or are) preferably selected from the group consisting of a TLR1 agonist, a TLR2 agonist, and a TLR4 agonist.

As used herein, the term “antigen” in particular refers to any molecule, moiety or entity capable of eliciting an immune response. This includes cellular and/or humoral immune responses. Depending on the intended function of the composition, one or more antigens may be included.

Particularly, the term “attached”, as used in the context of the present invention, preferably means “adsorbed”.

More preferably, said one or more antigens is (or are) selected from the group consisting of: lipopolysaccharide (LPS) or a derivative thereof, lipoteichoic acid (LTA), Pam(3)CysSK(4) ((S)-[2,3-5w(palmitoyloxy)-(2-RS)-propyl]-N-palmitoyl-(R)-Cys-(S)-Ser-(S)-Lys4-OH or Pam3-Cys-Ser-(Lys)), Pam3Cys (S-[2,3-bis(palmitoyloxy)-(2RS)-propyl]-N-palmitoyl-(R)-cysteine or tripalmitoyl-S-glyceryl cysteine), Cadi-05, ODN 1585, zymosan, synthetic triacylated and diacylated lipopeptides, MALP-2, tripalmitoylated lipopeptides, a compound having a 2-aminopyridine fused to a five membered nitrogen-containing heterocyclic ring, Polyriboinosinic-polyribocytidylic acid (poly IC), a CpG oligodeoxynucleotides (ODNs), monophosphoryl lipid A (“MPL”), an imidazoquinoline compound (e.g. an amide substituted imidazoquinoline amine), a benzimidazole derivative, a C8-substituted guanine ribonucleotide, an N7, C8-substituted guanine ribonucleotide, bacteria heat shock protein-60 (Hsp60), peptidoglycans, flagellins, mannuronic acid polymers, flavolipins, teichuronic acids, ssRNA (single stranded RNA), dsRNA (double stranded RNA), or a combination thereof.

Still more preferably, said one or more antigens is (or are) selected from the group consisting of proteins and peptides, and wherein the one or more antigens is preferably an alpha-toxin, more preferably Clostridium perfringens α-toxin or α-toxoid.

Most preferably, within the context of the invention, the TLR agonist described herein is a TLR4 agonist, in particular selected from LPS or a derivative thereof.

Thus, the amphiphile and/or the one or more antigens, as described herein, is (or are) in particular selected from the group consisting of LPS and LPS derivative, and wherein said LPS derivative is preferably selected from the group consisting of monophosphoryl lipid A (MPL), 3-O-deacylated monophosphoryl lipid A (3D-MPL), and Glucopyranosyl Lipid A (GLA).

Thus, in one preferred example the nanoparticle of the present invention is a nanoparticle coated with LPS or a derivative thereof (i.e. a LPS coated nanoparticle or a LPS derivative coated nanoparticle, respectively) and having a size within a range of from 50 to 200 nm, wherein said nanoparticle is composed of: a solid core consisting of PLGA or another biodegradable polymer, wherein optionally solvent molecules are included in the interior of the solid core; a shell of LPS or of a derivative thereof disposed over said solid core, wherein said derivative of LPS is selected from MPL, 3D-MPL, and GLA, and, optionally, one or more antigens attached to said amphiphile and/or said solid core.

The glucopyranosyl lipid A (GLA) is preferably a compound of formula (I):

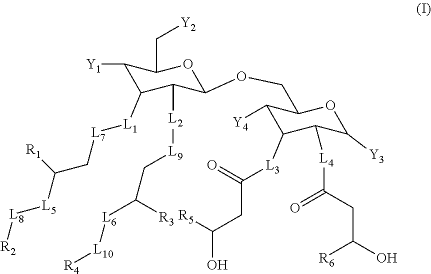

or a pharmaceutically acceptable salt thereof, wherein:
L₁, L₂, L₃, L₄, L₅ and L₆ are the same or different and are independently selected from —O—, —NH— and —(CH₂)—;
L₇, L₈, L₉ and L₁₀ are the same or different and are each independently either absent or —C(═O)—;
Y₁ is an acid functional group;
Y₂ and Y₃ are the same or different and are each independently selected from —OH, —SH, and an acid functional group;

### Y₄ is —OH or —SH;

R₁, R₃, R₅ and R₆ are the same or different and are independently C₈₋₂₀ alkyl; and
R₂ and R₄ are the same or different and are independently C₆₋₂₀ alkyl.

In particular, the GLA has the formula (I) set forth above or is a pharmaceutically acceptable salt thereof, wherein

Y₁ is preferably —OP(═O)(OH)₂; and/or
Y₂, Y₃ and Y₄ are preferably each —OH; and/or
R₁, R₃, R₅ and R₆ are the same or different and are independently C₈₋₁₃ alkyl; and/or
R₂ and R₄ are the same or different and are independently C₆₋₁₁ alkyl.

More preferably, the GLA mentioned herein is a compound of formula (II):

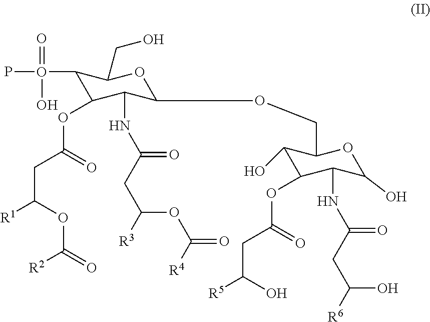

or a pharmaceutically acceptable salt thereof, wherein:
R¹, R³, R⁵ and R⁶ are C₁₁₋₂₀ alkyl; and
R² and R⁴ are C₁₂-C₂₀ alkyl.

According to one aspect, the GLA has the formula (II) set forth above, wherein R¹, R³, R⁵ and R⁶ are C₁₁₋₁₄ alkyl; and R² and R⁴ are C₁₂₋₁₅ alkyl. In a further, more specific, aspect, the GLA has the formula (II) set forth above wherein R¹, R³, R⁵ and R⁶ are C₁₁ alkyl; and R² and R⁴ are C₁₃ alkyl.

The GLA compounds described herein can be purchased or prepared according to methods known to those skilled in the art. Thus, the GLA having the formula (I) set forth above or, respectively, the GLA having formula (II) set forth above, may be either purchased or prepared by known organic synthesis techniques, such as e.g., described or referred to in the publication WO 2013119856 A1.

According to a further aspect, a method of producing the nanoparticle of the present invention is provided, wherein said method comprises or consists of the steps of:
a. adding (i) an organic solvent containing the biodegradable polymer to (ii) an aqueous phase containing the amphiphile, and
b. sonicating the combined organic solvent and aqueous phase at an energy sufficient to form a stable emulsion; and
c. evaporating the organic solvent from the stable emulsion;
   and wherein said method is also termed “the method of the present invention” hereinafter.

Preferably, the method of the present invention further comprises one or more of the following steps: separating the resulting nanoparticles from at least part of the remaining aqueous phase and preferably freeze drying the resulting nanoparticles and/or storing the resulting nanoparticles at a temperature of not more than 7° C.; and/or adding the one or more antigens or a composition comprising the one or more antigens to the remaining aqueous phase and/or the resulting nanoparticles.

In yet a further aspect, the invention is also directed to the nanoparticle of the present invention obtainable by the method of the present invention.

Preferably, the organic solvent mentioned herein is a nonpolar organic solvent, wherein said nonpolar organic solvent is in particular selected from the group consisting of ethyl acetate, methylene chloride, chloroform, tetrahydrofuran, hexafluoroisopropanol, and hexafluoroactone sesquihydrate.

According to another aspect, the invention further provides the nanoparticle of the present invention for use as an adjuvant or for use in a method for stimulating an immune response in a subject. It is understood that the term “adjuvant”, as mentioned herein, in particular refers to an “immunomodulatory agent”. Further, respectively, the term “adjuvant” used herein is in particular equivalent to the term “vaccine adjuvant”.

The term “subject” as used in the context of the present invention in particular relates to a human being or a non-human animal, wherein the non-human animal is preferably selected from the group consisting of swine, cattle, poultry, and companion animals.

The invention also provides the use of the nanoparticle of the present invention as an adjuvant for the manufacture of a vaccine, wherein the vaccine preferably comprises an antigen.

Also, the invention further provides a method for stimulating an immune response in a subject, wherein said method comprises the step of administering a composition comprising one or a plurality of the nanoparticles of the present invention to said subject.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Schematic presentation of nanoparticle preparation using an oil-in-water emulsification evaporation method.

FIG. 2: Study outline of in vivo studies with BALB/c mice.

FIG. 3: Influence of surfactant concentration on particle size of o/w-nanoparticles (mean diameter±SD, n=3).

FIG. 4: Influence of surfactant concentration on polydispersity of o/w-nanoparticles (mean±SD, n=3).

FIG. 5: Influence of surfactant concentration on zeta potential of o/w-nanoparticles (mean±SD, n=2).

FIG. 6: Loading rate of O/W-Nanoparticles (10 mg/ml) with 0.1 mg/ml Ovalbumin (mean±SD, n=3).

FIG. 7: Loading rate of O/W-Nanoparticles (10 mg/ml) with 0.1 mg/ml BSA (mean±SD, n=3).

FIG. 8: Influence of protein concentration on loading rate on (a) PVA (1%)-Nanoparticles, (b) TWEEN® 20 (1%)-Nanoparticles, (c) Sodium cholate (0.05%)-Nanoparticles and (d) SDS (0.01%)-Nanoparticles (mean±SD, n=3).

FIG. 9: In-vitro OVA release of o/w-NP in PBS at 37° C. (mean±SD; n=3).

FIG. 10: SDS-PAGE gels after staining with Coomassie Brilliant Blue, supernatants of o/w-NP formulations after incubation with toxoids and standard of toxoids; a) α-toxoid (E. coli): 1) Marker 2) PVA-NP 3) TWEEN® 20-NP 4) Sodium cholate-NP 5) SDS-NP 6) CTAB-NP 7) α-toxoid (E. coli) 0.065 mg/ml 8) α-toxoid (E. coli) 0.049 mg/ml 9) α-toxoid (E. coli) 0.0325 mg/ml 10) α-toxoid (E. coli) 0.016 mg/ml; b) α-toxoid (Clostridium perfringens): 1) PVA-NP 2) TWEEN® 20-NP 3) Sodium cholate-NP 4) SDS-NP 5) CTAB-NP 6) Marker 7) α-toxoid (Clostridium perfringens) 0.0054 mg/ml 8) α-toxoid (Clostridium perfringens) 0.0108 mg/ml 9) α-toxoid (Clostridium perfringens) 0.0161 mg/ml 10) α-toxoid (Clostridium perfringens) 0.0215 mg/ml.

FIG. 11: SDS-PAGE gels after staining with Coomassie Brilliant Blue, supernatants of o/w-NP formulations after incubation with toxoids, redispersed o/w-NP with SDS and DTT, and standard of α-toxoid (Clostridium perfringens): 1) Marker 2) PVA-NP supernatant 3) PVA-NP supernatant 4) PVA-NP redispersed (4×) 5) PVA-NP redispersed (1×) 6) α-toxoid (Clostridium perfringens) 0.0215 mg/ml 7) α-toxoid (Clostridium perfringens) 0.0161 mg/ml 8) α-toxoid (Clostridium perfringens) 0.0108 mg/ml 9) α-toxoid (Clostridium perfringens) 0.0054 mg/ml.

FIG. 12: Serum IgG-titers of mice after immunization (mean±SD, n=5-6; *p<0.05 compared to PBS and LPS+CpG, Kruskal-Wallis one way analysis of variance on ranks followed by Student-Newman-Keuls test).

FIG. 13: Serum IgG-titers of each mouse after study day 35 (n=5-6).

FIG. 14: Serum IgG-titers of mice after immunization (mean±SD; n=5-6).

FIG. 15: Serum IgG-titers of mice after immunization at SD 35 (mean±SD; n=5-6; *p<0.05 compared to I, $p<0.05 compared to VII, Kruskal-Wallis one way analysis of variance on ranks followed by Student-Newman-Keuls test).

FIG. 16: Serum IgG-titers of mice after immunization (mean±SD, n=4-5).

FIG. 17: Serum IgG-titers of mice after immunization at study day 35 (mean±SD, n=4-5; *p<0.05 compared to LPS-NP, Kruskal-Wallis one way analysis of variance on ranks followed by Student-Newman-Keuls test).

FIG. 18: Serum IgG-titers of mice after immunization at study day 35, CTAB-NP at different concentrations (mean±SD, n=4-5; *p<0.05 compared to LPS-NP, Kruskal-Wallis one way analysis of variance on ranks followed by Student-Newman-Keuls test).

FIG. 19: Serum IgG-titers of mice after immunization at study day 35, PVA-NP at different concentrations (mean±SD, n=5; *p<0.05 compared to LPS-NP, Kruskal-Wallis one way analysis of variance on ranks followed by Student-Newman-Keuls test).

FIG. 20: Serum IgG-titers of mice after immunization at study day 35, TWEEN® 20-NP at different concentrations (mean±SD, n=5; *p<0.05 compared to LPS-NP, Kruskal-Wallis one way analysis of variance on ranks followed by Student-Newman-Keuls test).

### EXAMPLES

### 1. Introduction/Objective

Novel vaccines consist of recombinant proteins that are safe to use, but often poorly immunogenic. In order to achieve a sufficient immune response adjuvants are essential. The requirements for an adjuvant are dependent on the type of immunogenic substance that is used as a vaccine. Despite the high demand only a few adjuvants are currently available for the use in humans and animals. At the moment, aluminum salts and oil-in-water (o/w)-emulsions are typically used as adjuvants. New adjuvants are needed for the challenges of finding vaccines for malaria, autoimmune diseases and cancer.

An innovative approach of developing modern adjuvants is the design of particulate antigen delivery systems. These, typically polymeric, particles that are in a size range of micro- and nanoparticles can be used as drug carrier systems. Such drug carriers can target antigen presenting cells, which is crucial for long-lasting immunity.

### 2. Materials and Methods

### 2.1. Substances

### 2.1.1. Poly(Lactic-Co-Glycolic Acid) (PLGA)

The polymer that was used to prepare nanoparticles (Manufacturer: Evonik; Trademark: RG 502 H; End group: Acid; Composition: Poly(D,L-lactide-co-glycolide) 50:50) has a composition of equal amounts of lactic acid and glycolic acid and they degrade in approximately one to two months in vitro.

The glass transition temperature of the PLGA copolymers is above 37° C. and the biodegradation occurs by non-enzymatic hydrolysis of the ester backbone.

PLGA is synthesized by ring-opening copolymerization of two different monomers, the cyclic dimers (1,4-dioxane-2,5-diones) of glycolic acid and lactic acid. Common catalysts used for this reaction include tin(II) 2-ethylhexanoate, tin(II) alkoxides or aluminum isopropoxide. PLGA is a FDA approved excipient (Drug Master File is registered with FDA), which is biocompatible and biodegradable.

### 2.1.2. Bovine Serum Albumin (BSA)

BSA was purchased from Sigma Aldrich (Munich, Germany). BSA consists of 583 amino acids and has a molecular weight of 66.4 kDa. The isoelectric point is 4.7. BSA was used as a model protein for the development of different micro- and nanoparticle formulations. It has already been widely used as a model protein for the preparation of micro- and nanoparticles due to its stability and low cost.

### 2.1.3. Ovalbumin (OVA)

OVA was obtained from Sigma Aldrich (Munich, Germany). OVA has a molecular weight of 45 kDa, consists of 386 amino acids, and has an isoelectric point of 4.86). OVA is the major protein in avian egg-white (60-65%), however its function is unknown. Nevertheless, OVA has been used as a model antigen in many vaccine studies, since it is a safe to use and well characterized immunogen. Here, we used OVA as a model protein for formulation experiments with nanoparticles and as a model antigen for in-vivo studies with mice.

### 2.1.4. α-Toxin

Clostridium perfringens is a Gram-positive anaerobe pathogen that causes gas gangrene. Every Clostridium perfringens strain possesses the gene encoding α-toxin. Formaldehyde α-toxins have already been used as experimental vaccines in humans and toxoid vaccines for sheep and goats are commercially available.

Two different C. perfringens α-toxoid antigens were kindly provided by Boehringer Ingelheim (Hannover, Germany). One antigen is α-toxoid derived from C. perfringens cell culture, the other antigen is E. coli derived α-toxoid. The Clostridium perfringens derived α-toxoid was inactivated with formaldehyde and neutralized with sodium bisulfite. E. coli express mutated which is therefore already inactivated α-toxoid. Both antigens were used for nanoparticle formulation experiments, as results are applicable to other recombinant proteins. α-Toxin has a molecular weight of 43 kDa.

### 2.1.5. Lipopolysaccharides (LPS)

LPS is a component of the cell wall of Gram-negative bacteria. LPS consists of three parts, a hydrophobic lipid (lipid A), a polysaccharide chain as the hydrophilic core and a hydrophilic O-antigenic polysaccharide side chain. LPS stimulates cells of the innate immune system by Toll-like receptor 4 (TLR4).

Lipopolysaccharides from salmonella enterica serotype abortusequi (LPS) and fluorescein isothiocyanate labeled lipopolysaccharide (FITC-LPS) were obtained from Sigma Aldrich (Munich, Germany). LPS was used to formulate PLGA-nanoparticles with surface adsorbed LPS (LPS-NP). These nanoparticles were used as adjuvants in in-vivo studies for this work. LPS was substituted with FITC-LPS to quantify the amount of LPS that was adsorbed on the particles.

### 2.1.6. Freund's Adjuvant

Freund's Adjuvant (CFA) and Incomplete Freund's Adjuvant (IFA) were purchased from Sigma Aldrich (Munich, Germany). CFA is an emulsion that consists of heat killed and dried Mycobacterium tuberculosis, paraffin oil and mannidemonooleate as the outer oil phase. The inner water phase contains the antigen, in this case OVA. IFA consists of paraffin oil and mannidemonooleate and lacks the bacteria. It also forms an emulsion with an inner water phase, which contains the antigen. For in-vivo study III in this work CFA and IFA were used as adjuvants. They were prepared by adding 2.5 ml of the oil phase to 2.5 ml of the water phase containing 0.01% OVA in PBS. The emulsion was formed by using an ULTRA TURRAX® (T 10 basic ULTRA TURRAX®, IKA®, Staufen, Germany) at 10000 rpm for 4 minutes. The resulting thick emulsion was tested by placing one drop of the emulsion on the surface of PBS. The drop of the emulsion was not allowed to disperse; if the drop disperses on the surface of PBS the emulsion is not stable and not suitable for injection.

### 2.1.7. Polyvinyl Alcohol (PVA)

Polyvinyl alcohol (PVA) (MOWIOL® 4-88, Kuraray Europe GmbH, Germany) was a gift from Kuraray Europe GmbH. PVA is a synthetic and water soluble polymer. After polymerization of vinyl acetate to polyvinyl acetate the hydrolysis of polyvinyl acetate results in PVA. PVA was used as a stabilizer in the outer water phase for the preparation of micro- and nanoparticles with the double emulsion method. It was also used as a surfactant for the preparation of nanoparticles with the o/w-emulsification-evaporation-method.

### 2.1.8. Polysorbate 20

Polysorbate 20 (TWEEN® 20) was purchased from Carl Roth (Karlsruhe, Germany). TWEEN® 20 is a polyoxyethylenesorbitan ester and has a molecular weight of 1225 g/mol. TWEEN® 20 is a nonionic surfactant with a critical micelle concentration (CMC) of approximately 0.05 mmol/l. It was used as a surfactant for the preparation of nanoparticles with the o/w-emulsification-evaporation-method.

### 2.1.9. Sodium Dodecyl Sulfate (SDS)

Sodium dodecyl sulfate (SDS) was obtained from Sigma Aldrich (Munich, Germany): SDS is an anionic surfactant with a molecular weight of 288 g/mol and a CMC of 8 mmol/l. It was used as a surfactant for the preparation of nanoparticles with the o/w-emulsification-evaporation-method and to linearize proteins in the polyacrylamide gel electrophoresis (SDS-PAGE).

### 2.1.10. Sodium Cholate

Sodium cholate was purchased from Sigma Aldrich (Munich, Germany). Sodium cholate is an anionic surfactant with a molecular weight of 431 g/mol and a CMC of approximately 12 mmol/l. It was used as a surfactant for the preparation of nanoparticles with the o/w-emulsification-evaporation-method.

### 2.1.11. Cetyltrimethylammonium Bromide (CTAB)

Cetyltrimethylammonium bromide (CTAB) was purchased from Carl Roth (Karlsruhe, Germany). CTAB is a cationic surfactant with a molecular weight of 364 g/mol and a CMC of approximately 0.9 mmol/l. It was used as a surfactant for the preparation of nanoparticles with the o/w-emulsification-evaporation-method.

### 2.1.12. Dithiothreitol (DTT)

Dithiothreitol (DTT) was obtained from Carl Roth (Karlsruhe, Germany). It has a molecular weight of 154 g/mol. DTT is a reducing agent. It was used in combination with SDS for quantification of adsorbed protein. DTT reduces disulfide bonds of the protein and SDS linearizes the protein. Furthermore, SDS causes the protein to desorb from the particle surface as it replaces the protein on the surface.

### 2.2. Oil-in-Water-Emulsification-Evaporation Method

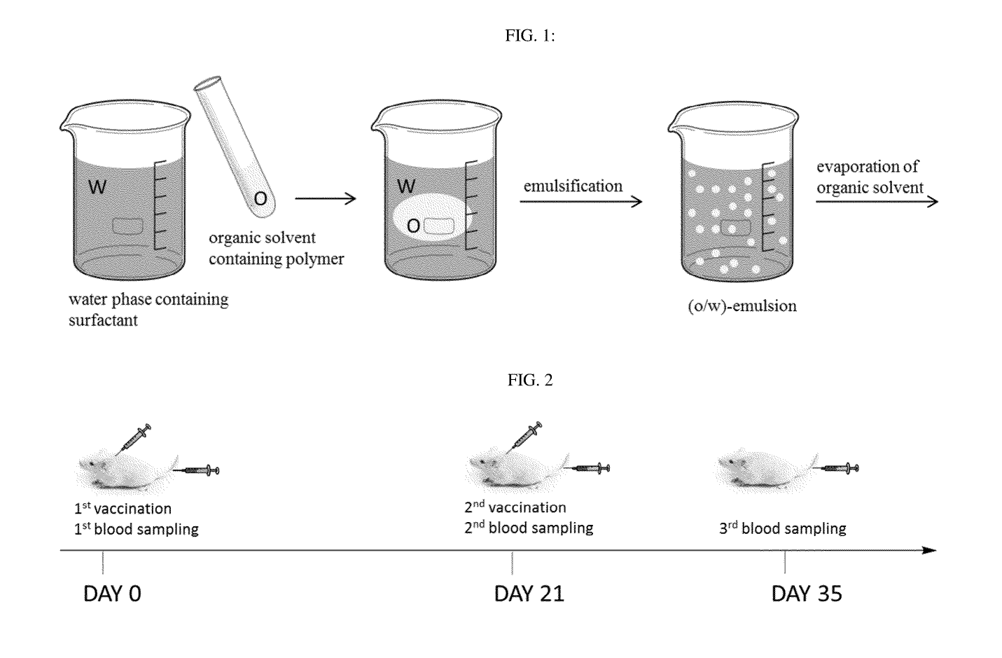

Biodegradable polymers, such as PLGA, can be used to prepare polymeric nanoparticles. For the purpose of preparing protein loaded nanoparticles an oil-in-water-emulsification-evaporation-method (FIG. 1) was used as previously described (Vauthier and Bouchemal, 2009; Wachsmann and Lamprecht, 2012). First an oil-in-water emulsion was formed. PLGA (20 mg/ml-100 mg/ml) dissolved in ethyl acetate acted as the oil phase. The outer water phase contained a surfactant (Table 1).

The organic phase was emulsified with the outer water phase by ultrasound using a Sonopuls HD 2200 sonicator (Bandelin electronic, Germany). The organic solvent of the resulting o/w-emulsion was then removed using a rotary evaporator at 45° C. under reduced pressure. The water insoluble PLGA precipitated as nanoparticles (o/w-NP).

TABLE 1
Surfactants used for the preparation of polymeric nanoparticles loaded
with proteins

| Surfactant | Concentration [g/100 ml] |
|---|---|
| PVA | 1; 0.3; 0.1 |
| TWEEN ® 20 | 1; 0.3; 0.1 |
| Sodium cholate | 0.1; 0.05; 0.01 |
| SDS | 0.1; 0.05; 0.01 |
| CTAB | 0.1; 0.05; 0.03; 0.01 |

2.2.1. Preparation of Nanoparticles with Adsorbed Proteins

Polymeric nanoparticles that were prepared as described previously (2.2) were incubated with a protein solution (BSA, OVA or Lysozyme) in various concentrations (0.1 mg/ml-2 mg/ml) for 3 hours on a horizontal shaker (Edmund Büler, Tübingen, Germany). Nanoparticles that were incubated with α-toxin were freeze-dried (see section 2.3) before incubation with the protein solution.

### 2.2.2. Preparation of LPS Loaded Nanoparticles

LPS loaded polymeric particles were prepared by an oil-in-water-emulsification-evaporation-method as described above (see section 2.2). PLGA (10 mg/ml) dissolved in ethyl acetate acted as the oil phase. LPS (1 mg/ml) was used in the outer water phase and no surfactant was necessary for the preparation of nanoparticles.

### 2.3. Freeze Drying of Nanoparticles

For stability studies of the nanoparticles that were loaded with the α-toxin, nanoparticles were freeze dried using a LYOVAC® GT2 (Steris, Germany). Trehalose (5% (m/V)) was added to the nanoparticle formulation as a cryoprotectant.

### 2.4. Analytical Methods

### 2.4.1. Particle Size Analysis

### 2.4.1.1. Photon Correlation Spectroscopy (PCS)

Particle size and polydispersity index (PDI) of nanoparticles were determined by photon-correlation spectroscopy (PCS) using a ZetaPlus particle sizer (Brookhaven Instruments Corporation, UK) at a fixed angle of 90° at 25° C. 100 μl of the nanoparticle sample was diluted with water in a UV-Cuvette macro (Brand GmbH, Germany). Each sample was measured in triplicate. Each measurement consisted of 5 runs with a duration of 1 minute each. The analysis was done using the Brookhaven Instruments Particle Sizing Software Version 3.88.

### 2.4.2. Loading-Rate of Protein

The loading rate of the o/w-NP and w/o/w-NP with the model proteins BSA and OVA was determined using a BCA-Assay. The NP-samples were centrifuged at 19000 rcf for 15 minutes and the supernatant was collected and measured by a BCA-Assay. Thus, an indirect quantification of the protein content was performed. The encapsulation rate of the microparticle samples were also measured indirectly. After filtration of the obtained microparticle suspension, the protein content of the filtrate was investigated using a BCA-Assay. The loading rate of the o/w-NP with the α-toxin was handled as the o/w-NP samples with BSA and OVA. However, the supernatant was not examined by BCA-Assay, but SDS-PAGE gel electrophoresis (see 2.4.2.2).

### 2.4.2.1. BCA-Assay

Protein contents of the micro- and nanoparticle samples were measured by a BCA-Assay (ROTI®-Quant universal assay, Carl Roth, Germany). Cu²⁺ ions are being reduced to Cu¹⁺ ions by protein bonds. The principle of the BCA-Assay is that bicinchoninic acid forms an intense purple complex with cuprous ions (Cu¹⁺) in alkaline environment. 100 μl of the samples and standards were placed in a 96-well plate (PerkinElmer, Waltham, USA) and mixed with 100 μl of reagent solution of the BCA-Assay Kit. After incubation at 37° C. for 30 minutes the absorbance was measured at 490 nm using a plate reader (1420 Multilabel Counter Victor3 V, PerkinElmer, USA).

### 2.4.2.2. 2 SDS-PAGE Gel Electrophoresis

To quantify the α-toxin content on the o/w-NP a SDS-polyacrylamide gel electrophoresis (PAGE) was performed. SDS-PAGE is widely used to separate proteins according to their molecular weight. The samples were mixed with “Laemmli-buffer” containing SDS to linearize the protein and additionally to apply a negative charge to each protein. The buffer also contained glycerol to increase the density of the sample and Bromphenol blue as a tracking dye. The sample was further heated to 95° C. for 5 minutes using a heating block (THERMOMIXER® comfort, Eppendorf, Germany) and 2-mercaptoethanol was added to reduce disulfide linkages. SDS-PAGE was performed using a MINI-PROTEAN®-system (Bio-Rad Laboratories, USA). The self-prepared separating gels had an acrylamide content of 12% and the stacking gels had an acrylamide content of 4%. Polymerization was initiated by adding ammonium persulfate and tetramethylethylenediamine. After adding the samples to the gels an electric field was applied, which led to the negatively charged proteins migrating to the positive electrode (anode). The gel was run for 2 h at 20 mA. The gels were then stained with Coomassie Brilliant Blue for 8 h and bleached with water, methanol and acetic acid afterwards. To calculate the protein content, α-toxin samples with a known concentration were used as a standard. Further a marker (ROTI®-Mark Standard, Carl Roth, Germany) was used to estimate the molecular weight of the separated proteins. To quantify the α-toxin content the stained gels were placed on a REFLECTA® L 300 light panel (Intas Science Imaging Instruments GmbH, Göttingen, Germany) and photographed with an Intas camera system (Intas Science Imaging Instruments GmbH, Göttingen, Germany) and then a densitometric analysis was put out with ImageJ analysis system.

### 2.4.3. Release Test

The in-vitro dissolution tests were all carried out in PBS. A defined amount of the dried microparticle sample was suspended in a conical flask with PBS. The conical flask was incubated in a shaking water bath (GFL, Burgwedel, Germany) at 37° C. at 80 rpm. Samples were withdrawn at various times for the analyses of drug release. The protein content was determined as described above. The in-vitro dissolution tests of the nanoparticle samples were carried out in Eppendorf cups. 100 μl of nanoparticle suspension was mixed with 900 μl of PBS. Samples were withdrawn at various times for the analyses of drug release. The protein content was determined as described above.

### 2.4.4. Zeta Potential

The measurement of the zeta potential was carried out using a ZetaPlus particle sizer (Brookhaven Instruments Corporation, UK). The analysis was done using the Brookhaven Instruments Zeta Potential Analyzer Software Version 3.54.

### 2.5. In Vivo Experiments

The immune response of the o/w-NP and the LPS-NP was determined by animal experiments using BALB/c mice. As a model protein OVA was used. This is a well-established in-vivo model to simulate adjuvant effects.

### 2.5.1. BALB/c Mouse

The BALB/c mice were purchased from Charles River (Sulzfeld, Germany). The BALB/c mouse is an albino and laboratory bred strain. All experiments were carried out in the “HausfürExperimentelleTherapie” (HET) in Bonn. The mice were 4 weeks old and weighed 25-35 g. Only male mice were used. The animal trial began after an acclimation period of seven days. The mice were fed with autoclaved standard food (SSNIFF®, Soest, Germany) and water ad libitum. 3-5 mice were kept in one cage and the cages were changed once a week. Individually Ventilated Cages (IVCs) were used in this study. The cages were kept in a room with a temperature of 22° C. and an overpressure of 150 Pa. The relative humidity was approximately 50-60%.

### 2.5.2. Study Outline

The influence of OVA loaded carriers was investigated using a mouse-model. Therefore, five in-vivo studies were conducted. The immune response of nanoparticles and different adjuvant formulation was tested. PLGA particles have already shown that they can have an effect on the immune response (Gutierro et al., 2002a; Waeckerle-Men and Groettrup, 2005). In the presented work, we investigated different adjuvant formulations and the effect of different o/w-NP formulations. For all in-vivo trials the animals were immunized two times and blood samples were drawn three or four times (FIG. 2).

All animal experiments started with marking the mice. Therefore, an ear puncher that was kindly provided by the HET was used. Depending on the number of mice in one cage, either 4 mice or 2 mice were marked.

The immunization was performed subcutaneously in the neck using a 23 G needle (B. Braun, Melsungen, Germany) on study day (SD) 0 and on SD 21. The OVA solution or nanoparticle formulation was drawn up in the syringe and 100 μl was injected in each mouse. The needle was always changed for each mouse. The blood withdrawal from the tail was done using a 22 G needle (B. Braun, Melsingen, Germany) and micro hematocrit tubes (Brand, Wertheim, Germany). Approximately 50-100 μl blood was drawn and put in an Eppendorf cup, while the mouse was fixed in a restrainer. The blood was stored at room temperature for approximately one hour. Then it was stored at 4° C. for 24 hours. Afterwards, it was centrifuged at 19000 rcf for 15 min. using a Hermle Z 233 M-2 (HermleLabortechnik, Wehingen, Germany). Then approximately 10-20 μl of the blood serum (supernatant) was collected and stored at −20° C.

The injection site of the animals was monitored daily after the subcutaneous injection for three days and then once a week. Several abort criteria were set to guarantee minimal distress for the animals. If any of the following signs were observed, the experiment with this animal was terminated by euthanizing:

a. abnormal body posture

b. loss of mobility

c. visible inflammations

d. weight loss ≧20%

### 2.5.2.1. In Vivo Testing of Different Adjuvants

In the animal experiment (Table 2) different adjuvant formulations were tested in regard to the immune response. The OVA concentration was 10 μg per dose in all groups. PBS with OVA acted as a control group. The second group contained OVA and CFA for the first immunization and IFA for the second immunization. The adjuvant protein emulsion was formed as described above (2.1.6). This group acted as a positive control, since the intense immune response in mice after the administration of CFA is widely known. The third group and fourth group were the experimental groups. Here we compared the immune response of LPS-NP combined with CpG with soluble LPS combined with CpG. Since CFA and IFA are highly toxic and dangerous, the mice in this group were anesthetized with isofluran (FORENE®, Abbott, Germany) during the immunization. The blood withdrawal took place on SD 0, SD 21 and SD 35.

TABLE 2
Experimental groups

| | | | | Number |
|---|---|---|---|---|
| Group | Formulation | Ovalbumin | Adjuvant | of mice |
| I | PBS | 10 μg | — | 5 |
| II | PBS | 10 μg | CFA/IFA (50%) | 6 |
| III | LPS (1 μg)-NP | 10 μg | 5 μg CpG | 6 |
| IV | PBS | 10 μg | 5 μg CpG; 1 μg LPS | 5 |

### 2.5.2.2. Lipopolysaccharide Loaded Nanoparticles and Ovalbumin Loaded Nanoparticles in Mice

In the in-vivo study (Table 3) the effect of five different o/w-NP formulations on the immune response was tested. The OVA concentration was 10 μg per dose in all groups and LPS-NP (LPS concentration 1 μg per dose) coupled with CpG (5 μg per dose) was used as an adjuvant in all groups. The first group was the control group, containing free OVA and the adjuvant formulation. The eighth group was also a control group, containing free OVA without the adjuvant formulation. The other groups were the experimental groups. OVA was loaded on nanoparticles as described above (see 2.2.1). The resulting nanoparticle formulations were tested. Group II contained the TWEEN® 20 (1%)-NP, Group III the PVA (1%)-NP, Group IV the Sodium cholate (0.05%)-NP, Group V the SDS (0.01%)-NP and Group VI the CTAB (0.05%)-NP. The blood withdrawal was on SD 0, SD 21 and SD 35.

TABLE 3
Experimental groups

| | | | | Number of |
|---|---|---|---|---|
| Group | Formulation | Ovalbumin | Adjuvant | mice |
| I | LPS-NP | 10 μg | LPS (1 μg)-NP; 5 μg CpG | 6 |
| II | TWEEN ® 20 (1%)-NP | 10 μg | LPS (1 μg)-NP; 5 μg CpG | 6 |
| III | PVA (1%)-NP | 10 μg | LPS (1 μg)-NP; 5 μg CpG | 6 |
| IV | Sodium cholate | 10 μg | LPS (1 μg)-NP; 5 μg CpG | 6 |
| | (0.05%)-NP | | | |
| V | SDS (0.01%)-NP | 10 μg | LPS (1 μg)-NP; 5 μg CpG | 5 |
| VI | CTAB (0.05%)-NP | 10 μg | LPS (1 μg)-NP; 5 μg CpG | 6 |
| VII | PBS | 10 μg | — | 6 |

### 2.5.2.3. Influence of Nanoparticle Concentration on Immune Response in Mice

In the in-vivo study (Table 4) the effects of the nanoparticle concentration on the immune response was investigated. An OVA solution in PBS coupled with LPS-NP (LPS concentration 1 μg per dose) and CpG (5 μg per dose) was used as an adjuvant in all groups, including the control group. The nine experimental groups had all the same amount of OVA in their formulation (10 μg per dose). CTAB (0.05%)-NP, TWEEN® 20 (1%)-NP and PVA (1%)-NP were tested in different concentrations. The particles were prepared as described before (see 2.2.1) with slight deviations. Briefly, the PLGA amount was changed (100 mg/ml and 4 mg/ml instead of 20 mg/ml) and the sonication duration was adjusted, in order to obtain comparable nanoparticle sizes. The blood withdrawal was performed on SD 0, SD 21 and SD 35.

TABLE 4
Experimental groups

| | | PLGA | | |
|---|---|---|---|---|
| | | concentration | | Number |
| Group | Formulation | [mg/ml] | Adjuvant | of mice |
| I | LPS-NP | — | LPS (1 μg)-NP; 5 μg CpG | 5 |
| II | CTAB (0.05%)-NP | 2 | LPS (1 μg)-NP; 5 μg CpG | 5 |
| III | TWEEN ® 20 (1%)-NP | 2 | LPS (1 μg)-NP; 5 μg CpG | 5 |
| IV | PVA (1%)-NP | 2 | LPS (1 μg)-NP; 5 μg CpG | 5 |
| V | CTAB (0.05%)-NP | 10 | LPS (1 μg)-NP; 5 μg CpG | 5 |
| VI | TWEEN ® 20 (1%)-NP | 10 | LPS (1 μg)-NP; 5 μg CpG | 5 |
| VII | PVA (1%)-NP | 10 | LPS (1 μg)-NP; 5 μg CpG | 5 |
| VIII | CTAB (0.05%)-NP | 50 | LPS (1 μg)-NP; 5 μg CpG | 4 |
| IX | TWEEN ® 20 (1%)-NP | 50 | LPS (1 μg)-NP; 5 μg CpG | 5 |
| X | PVA (1%)-NP | 50 | LPS (1 μg)-NP; 5 μg CpG | 5 |

### 2.5.2.4. IgG-ELISA

The blood samples of the in-vivo studies were left to thaw overnight in a fridge (−20° C.) and then measured using the Mouse Anti-Ovalbumin IgG kit from Alpha Diagnostics (San Antonio, USA). The ELISA was carried out as described in the manual (Instruction Manual No. M-600-105-OGG). Briefly, after preparing a “washing solution” as well as a solution to dilute the samples, the 96-well plate that was covered with OVA was washed using the “washing solution”. Meanwhile, all samples were appropriately diluted (100-500000×). 100 μl of standards and samples were added to the plate and incubated for 60 minutes to bind the IgG on the immobilized OVA on the wells. After several washing steps, 100 μl of an IgG-specific antibody conjugated with horseradish peroxidase (HRP) was added and incubated for 30 minutes. The IgG-specific antibody was bound to IgG. After several washing steps the excess of the free IgG-specific antibody conjugated with HRP was washed off. Then 100 μl of 3,3′,5,5′-Tetramethylbenzidine (TMB) was added as a chromogenic substrate. The HRP reacted with the TMB to a blue colored product. TMB was oxidized by HRP, resulting in a diimine. By adding 100 μl sulfuric acid (1%) the TMB turned yellow. The absorbance was then measured at 450 nm using a plate reader (1420 Multilabel Counter Victor3 V, PerkinElmer, USA). The amount of mouse IgG in the samples was calculated relative to anti-ovalbumin reference calibrators. The results were indicated as IgG Antibody Activity Units (U*ml⁻¹).

### 2.6. Statistical Analysis

The statistical analysis was carried out using Sigmastat 2.0 Software. Statistical difference was investigated by Kruskal-Wallis Anova on Ranks followed by multiple comparisons with Student Newman-Keuls test. The data was expressed as mean±SD, p<0.05 was considered to be significant.

### 3. Results

3.1. Nanoparticles Prepared with Oil-in-Water-Emulsification-Evaporation Method

As shown previously, nanoparticles prepared by the double-emulsion method do not encapsulate the hydrophilic drug inside at a particle size below 600 nm. The hydrophilic drug is adsorbed at the surface. Hence, preparing nanoparticles using a simpler approach is beneficial in terms of stability of the hydrophilic drug, since it is exposed to shear stress, heat and to an organic solvent, when applying the double-emulsion method.

Therefore, nanoparticles were prepared using an oil-in-water-emulsification-evaporation method. The “blank” PLGA-NP were then incubated with the hydrophilic drug, BSA, OVA or α-toxin.

### 3.1.1. Physicochemical Characterization of Nanoparticle Properties

### 3.1.1.1. Influence of Surfactants on Particle Size and Polydispersity

Five different surfactants were used to prepare the PLGA-NP. The nonionic surfactants PVA and TWEEN® 20, the anionic surfactants SDS and sodium cholate and the cationic surfactant CTAB were used for the preparation. As the protein was adsorbed after the preparation of the PLGA-NP it was anticipated that modified surface properties of the PLGA-NP, as a result of different surfactants, would have an effect on the loading rates.

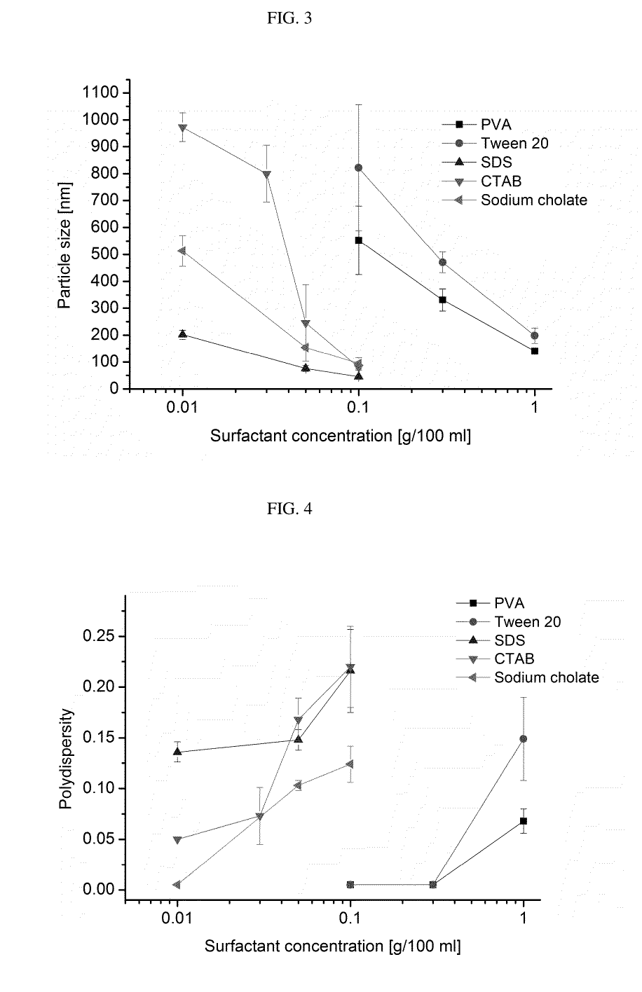

Higher amounts of surfactant led to a smaller particle size of the PLGA-NP (FIG. 3). The emulsion is stabilized with surfactants during the emulsification process via ultrasonication. A higher amount of surfactant can stabilize smaller droplets compared to lower amounts of surfactants. This ultimately yields smaller particles.

The goal was to obtain nanoparticles in the size range of 100 nm-200 nm (mean diameter). This was possible for all formulations, using sufficient amount of the respective surfactant. For PVA-NP and TWEEN® 20-NP a surfactant concentration of 1% w/v was used to get nanoparticles below 200 nm, SDS-NP in the desired particle size range were obtained using 0.01% w/v SDS and for the CTAB-NP 0.1% w/v CTAB was necessary. Sodium cholate-NP had a particle size of 154 nm±10 nm, when using 0.05% w/v sodium cholate.

As another important aspect, the polydispersity of the nanoparticle formulations was investigated. As mentioned earlier, a high polydispersity indicates that the particle distribution is not monomodal. A polydispersity index above 0.05-0.1 suggests a bimodal particle size distribution.

The polydispersity index increases with higher amounts of surfactants and consequently increases for smaller particles (FIG. 4).

Polydispersity values close to 0.005 indicate monomodal particle size distributions. This could only be observed for particles above 300 nm. Higher amounts of surfactants were evidently sufficient to obtain small particles, but failed to obtain nanoparticles with a monomodal particle size distribution at a size range of 100 nm-200 nm.

### 3.1.1.2. Zeta Potential of Nanoparticles

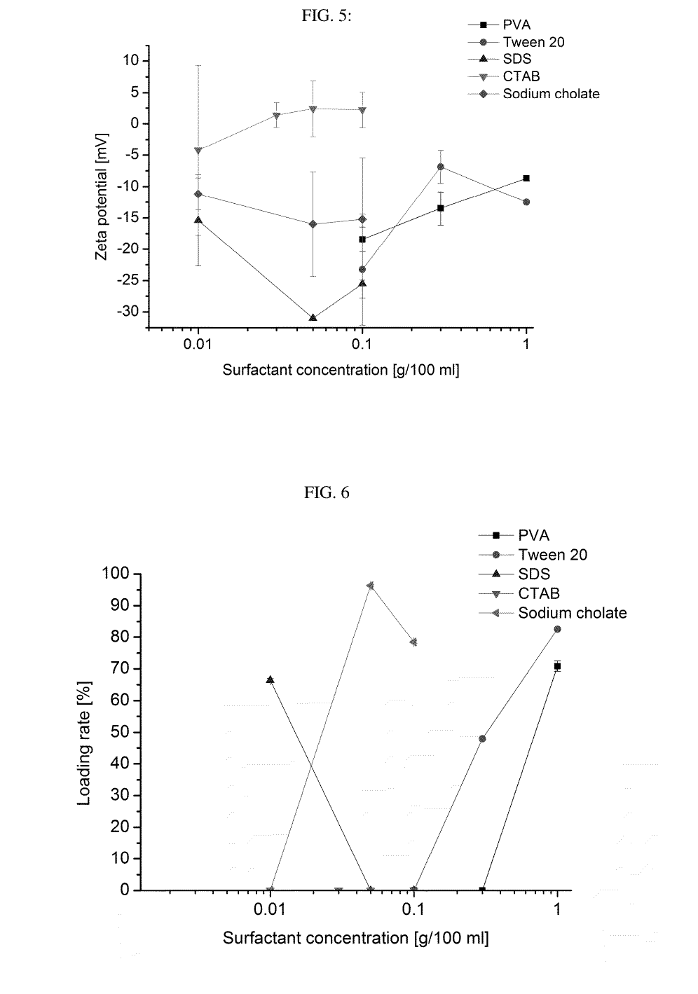

The surface properties of the PLGA-NP were characterized by measurement of the zeta potential as described in 2.4.4. The zeta potential changed for each surfactant and showed values of −7 mV-−25 mV for the nonionic and anionic surfactants (FIG. 5). The CTAB-NP had a zeta potential of −4 mV to 2 mV.

### 3.1.1.3. Loading Rate of Adsorbed Proteins on Nanoparticles

The prepared PLGA-NP were further tested regarding their potential to adsorb protein onto the surface, using OVA or BSA as model proteins. All formulations, using PVA, TWEEN® 20, SDS, sodium cholate, and CTAB were prepared as described in chapter 2.2.1 and investigated.

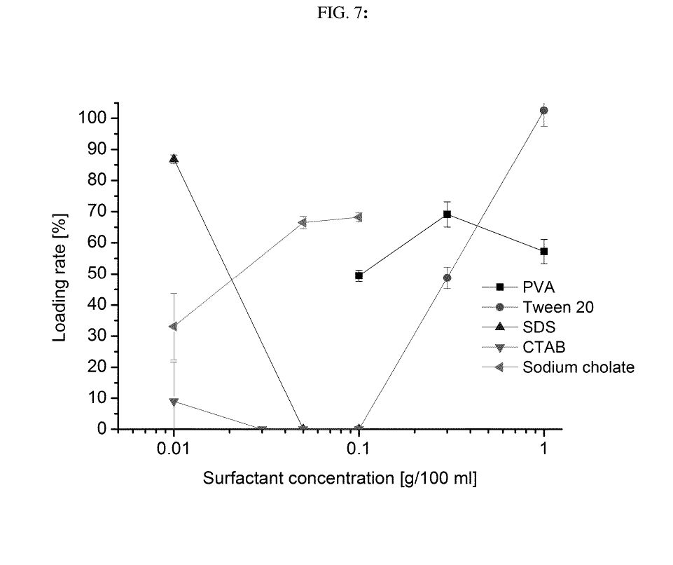

OVA showed a high loading to the surface of the nanoparticles at a size below 200 nm (FIG. 6).

The amount of OVA adsorbed at the surface of the NP increased for smaller particles, when using PVA and TWEEN® 20 as surfactants. However, this was not the case for the SDS- and sodium cholate-formulations. Especially for SDS-NP the loading rate decreases to 0%, meaning that no protein is adsorbed to the surface at a concentration of 0.1% SDS. Even though the particle size of the SDS-NP is smaller when using 0.1% SDS (46 nm±2 nm) instead of 0.01% (202 nm±16 nm), the loading rate decreases. This is most likely due to the fact that the SDS is repulsing the protein on the surface of the PLGA-NP. At low concentrations of SDS OVA is able to adsorb at the surface, but not at high SDS concentrations. A similar, but not as drastic effect, can be seen when using sodium cholate as a surfactant. The highest loading of OVA can be observed for sodium cholate-NP prepared with 0.05% sodium cholate. Increasing the sodium cholate concentration to 0.1% results in a lower loading rate, however the difference is not as substantial as for the SDS-NP. Like for all other tested surfactants, the particle size also decreases with higher surfactant concentrations, which, in theory, should lead to an increase in loading rate as smaller particles provide a larger surface available for adsorption. This means, that surface properties such as zeta potential and hydrophobicity are of high importance as the surface area is apparently not itself solely responsible for the protein adsorption.

For the nonionic surfactants PVA and TWEEN® 20 the loading rate follows the understanding that higher loading rates can be observed for smaller particles. Here, increasing surfactant concentrations yield smaller particles that have a bigger surface area (Table 5). A high amount of surfactant does not hinder OVA adsorption at the surface, when using the nonionic surfactants PVA and TWEEN® 20. Both appear advantageous compared to the anionic surfactants SDS and sodium cholate.

CTAB was used as a cationic surfactant. Surprisingly, OVA does not adsorb at the surface of CTAB-NP. Independent of the size of the CTAB-NP and the amount of CTAB used, a loading of the CTAB-NP with OVA could not be obtained.

TABLE 5
Surface area of o/w-NP prepared with different surfactants at various
concentrations

| Surfactant | Surface area of 1 g w/o/w-NP [nm²] | | | | |
|---|---|---|---|---|---|
| concentration | | TWEEN ® | | Sodium | |
| [g/100 ml] | PVA-NP | 20-NP | SDS-NP | cholate-NP | CTAB-NP |
| 1 | 35.5 × 10¹⁸ | 25.2 × 10¹⁸ | | | |
| 0.3 | 15.1 × 10¹⁸ | 10.6 × 10¹⁸ | | | |
| 0.1 | 9.1 × 10¹⁸ | 6.1 × 10¹⁸ | 110 × 10¹⁸ | 53.0 × 10¹⁸ | 62.5 × 10¹⁸ |
| 0.05 | | | 65.7 × 10¹⁸ | 32.4 × 10¹⁸ | 20.4 × 10¹⁸ |
| 0.035 | | | | | 6.25 × 10¹⁸ |
| 0.01 | | | 24.8 × 10¹⁸ | 9.75 × 10¹⁸ | 5.14 × 10¹⁸ |

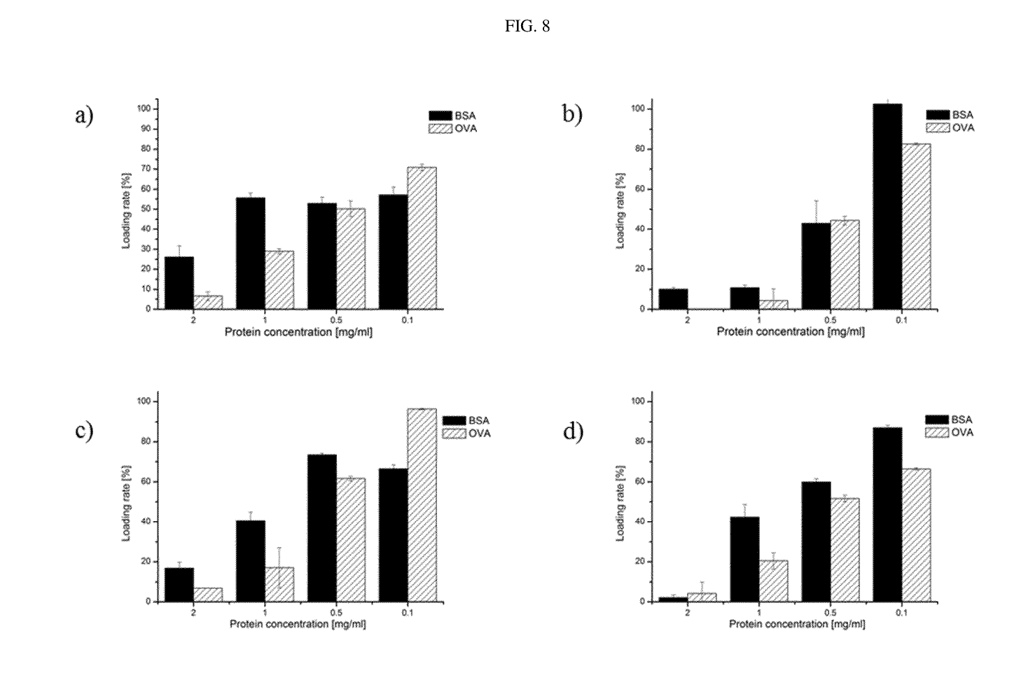

Another model protein that was used in this work was BSA. The results regarding the loading rate of BSA onto the surface of PLGA-NP are similar to those obtained when using OVA as a protein. For the nonionic surfactants PVA and TWEEN® 20 the loading rate increases with a bigger surface area. PVA-NP do show a slight decrease of the loading rate for 1%, here the high amount of PVA affects the loading with BSA negatively. For TWEEN® 20-NP and sodium cholate-NP a higher loading of BSA was observed for smaller nanoparticles, which have a bigger surface area (FIG. 7).

SDS-NP showed a similar behavior when comparing the loading of BSA and OVA. Only at a concentration of 0.01% a loading with BSA could be observed. This may also be the effect of SDS competing with BSA on the surface of the nanoparticles, making an adsorption of BSA at high SDS concentrations impossible.

A loading with BSA on CTAB-NP could also only be observed at the lowest used CTAB concentration. This is an improvement compared to the loading with OVA, where a loading was not observed. The low loading rate at 0.01% CTAB combined with the fact that no loading was possible at higher CTAB concentrations suggests that CTAB competes with the proteins at the surface of the nanoparticles, making a loading with the proteins unlikely.

When comparing the nanoparticle formulations that produced the highest loading with the proteins at different protein concentrations, it can be observed that BSA tends to be more adsorbed at the nanoparticle surface compared to OVA (FIG. 8). However, this was not observed for all formulations, but for the majority of nanoparticle formulations. The CTAB-NP were not included for the comparison of OVA and BSA as a loading with protein on the CTAB-NP surface was not observed. BSA causes a lower surface tension in PBS than OVA (Table 9). Therefore, it can be assumed that BSA, compared to OVA, accumulates more on interfaces, which could explain why the loading rate of BSA is higher on most o/w-NP formulation. However, the surface-active properties of the protein are not the only determining factors for the adsorption of the protein onto the nanoparticle surface, hydrophobic and ionic interaction also impact the ability of the protein to adsorb at the nanoparticle surface. The surfactant used for the preparation of the o/w-NP causes a modified nanoparticle surface. Thereby, the loading rate of each protein onto the nanoparticle surface must be tested for each o/w-NP formulation as the surface properties of the o/w-NP surface changes with each formulation leading to different intensities of ionic and hydrophobic interactions with the protein.

The studies regarding release kinetics, stability, morphology, cell interaction and in-vivo experiments were conducted using nanoparticle formulations that had a relatively good loading rate combined with a small particle size (Table 6).

TABLE 6
Particle formulations used for further studies

| Nanoparticle | Loading rate (OVA | |
|---|---|---|
| formulation (10 mg/ml) | 0.1 mg/ml) [%] | Mean diameter [nm] |
| PVA (1%) | 71 ± 2 | 141 ± 9 |
| TWEEN ® 20 (1%) | 83 ± 2 | 198 ± 28 |
| Sodium cholate (0.05%) | 96 ± 1 | 154 ± 10 |
| SDS (0.01%) | 66 ± 2 | 202 ± 16 |
| CTAB (0.05%) | 0 | 246 ± 142 |

### 3.1.1.4. Release Profile of Protein Loaded Nanoparticles

The in-vitro release profile of the o/w-nanoparticle formulations (Table 6) prepared using different surfactants showed similar results to the nanoparticles prepared with the double emulsion method, using only PVA.

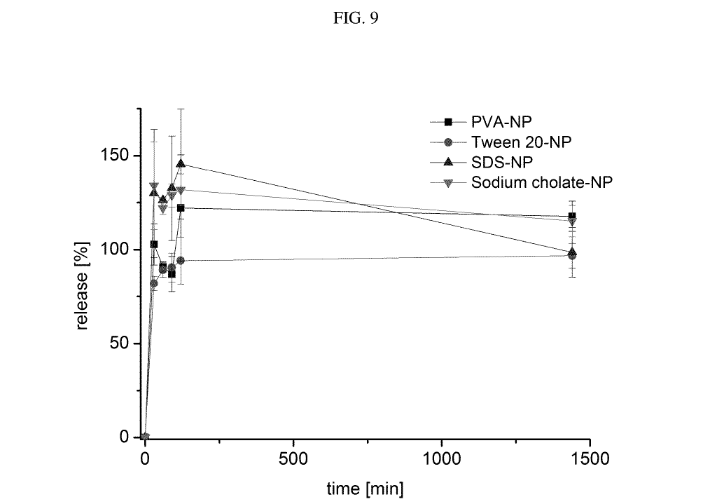

The o/w-NP showed an immediate release of OVA in PBS (FIG. 9) within 30 minutes. The Protein is just adsorbed at the surface, making a fast release possible. A release of about 100% was achieved for all nanoparticle formulations. Therefore, the surface modification with the surfactants has no influence on the release profile.

The results of the release profile are slightly defective as the release of the protein was over 100%. This can be attributed to the fact that the nanoparticle samples were directly measured after sampling. The samples were drawn at a temperature of 37° C., the increased temperature of the nanoparticle samples compared to the standards led to an increase in the absorption of the detected bicinchoninic acid, which is measured when determining the protein content with a BCA-assay.

### 3.1.1.5. Characterization of α-Toxoid Loaded Nanoparticles

Recombinant proteins are in high demand in vaccine research as they pose as a safer choice compared to live, attenuated vaccines. Challenges regarding the immunogenicity have been discussed earlier (2.1). In addition to those challenges, recombinant proteins are rarely of a high purity making the formulation development difficult. Residual host cell impurities like proteins and DNA can interact with the nanoparticles leading to aggregation.

In this work, α-toxoid from E. coli and α-toxoid from Clostridium perfringens, which can be used as vaccines to protect animals against gas gangrene, was tested regarding its compatibility and loading rate with o/w-NP.

The o/w-NP showed promising properties concerning loading rate, stability and compatibility when model proteins like BSA and OVA were used. To test if the o/w-NP are also suitable for the formulation with recombinant proteins, α-toxoid from E. coli was tested. Furthermore, α-toxoid derived from Clostridium perfringens was also tested.

The PLGA o/w-NP were loaded with α-toxoid as described in section 2.2.1. Before the proteins were incubated with the o/w-NP the nanoparticles were freeze dried as previously described (2.3). The protein solution was then incubated for 3 hours with the freeze dried nanoparticles. PLGA nanoparticles were used in a concentration of 12.5 mg/ml in the loading experiment.

The α-toxoid from E. coli was added to yield a final concentration of 0.065 mg/ml and the α-toxoid from Clostridium perfringens was added to yield a concentration of 0.0215 mg/ml in the nanoparticle formulation.

The α-toxoid from E. coli was easier to formulate with nanoparticles, except for the CTAB-NP all formulations formed stable nanoparticle suspension (Table 7). Visual aggregation after blending of nanoparticle formulation and protein was considered as “instable”.

The α-toxoid from Clostridium perfringens was not stable when blending with the ionic o/w-NP. Precipitation was observed during incubation of the α-toxoid from Clostridium perfringens with SDS-NP, sodium cholate-NP, and CTAB-NP. The formulation was physically stable when the nonionic PVA or TWEEN® 20 was used for the preparation of the o/w-NP.

TABLE 7
Compatibility of different O/W-Nanoparticle formulations with α-toxoid
from E. coli and α-toxoid from Clostridium perfringens.
(+) indicates that blend of nanoparticle formulation and protein solution
is suitable for further testing. (−) indicates that precipitation occurred
during blending process (n = 3-4).

| | | α-toxoid (Clostridium |
|---|---|---|
| Nanoparticle formulation | α-toxoid (E. coli) | perfringens) |
| PVA (1%) | + | + |
| TWEEN ® 20 (1%) | + | + |
| Sodium cholate (0.05%) | + | − |
| SDS (0.01%) | + | − |
| CTAB (0.05%) | − | − |

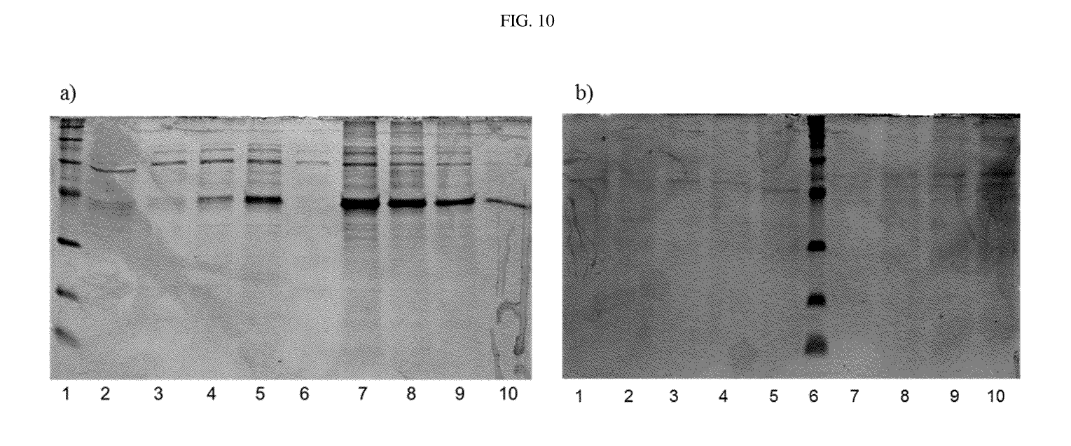

The protein is not extensively purified, which is visible in the SDS-PAGE gels (FIG. 10). The α-toxoid from E. coli has a purity of approximately 50% and the α-toxoid from Clostridium perfringens has a purity of approximately 25%. As the proteins are unpurified a BCA-assay cannot give reliable results concerning the adsorption of the toxoids on the surface, because the total amount of protein is measured when applying a BCA-assay, the method is not specific for α-toxoid. Therefore, SDS-PAGE was used to measure the amount of the different proteins on the surface of the nanoparticles. An indirect method was used, as mentioned previously (2.4.2).

It must be noted that some o/w-NP formulations were unstable when blending with the toxoids. Precipitation was observed for some formulations, this is important when examining the gels, as an indirect method was applied measuring the protein in the supernatant of the o/w-NP after incubation. In case of precipitation, the toxoid formed agglomerates with the nanoparticles, and the supernatant did not contain any toxoid. Consequently, even if no protein was detected in the supernatant, which would normally mean that 100% is adsorbed at the nanoparticle surface, adsorption of the toxoid to the nanoparticle surface did not take place, if precipitation occurred during the incubation. In this context, the supernatant of CTAB-NP, for example, contained no toxoid at all (FIG. 10), and this is attributed to the fact that CTAB-NP agglomerated with the protein solutions (Table 7). CTAB-NP were therefore considered not to be suitable for either, α-toxoid from E. coli or α-toxoid from Clostridium perfringens.

The o/w-NP using anionic surfactants for the preparation (SDS and sodium cholate) also precipitated when mixing with the α-toxoid from Clostridium perfringens. Hence, these formulations are not appropriate for this protein. However, the formulations with α-toxoid from E. coli were successful regarding their physical stability. Here, the amount of protein in the supernatant could be used to calculate the loading rate of the toxoid to the nanoparticle surface. For the o/w-NP using nonionic surfactants (PVA and TWEEN® 20) formulations with both toxoids were physically stable, consequently supernatant was used to determine the amount of toxoid on the o/w-NP surface.

The SDS-PAGE gels were visualized with an Intas camera system and a densitometric analysis was performed using the imageJ software. The experiments in which the loading of o/w-NP with α-toxoid from E. coli was tested could be easily evaluated, as the proteins from the vaccine formulation were clearly separated and had a high enough concentration to be sufficiently visible for a quantitative measurement.

The o/w-NP prepared with the nonionic surfactants PVA and TWEEN® 20 showed the highest loading with α-toxoid from E. coli with loading rates of 94%±6% and 96%±1%, respectively (Table 8). The sodium cholate-NP also showed a high loading rate of 80%±1% and the SDS-NP had a loading rate of 41%±10%.

TABLE 8
Loading rate of o/w-NP with α-toxoid (E. coli)

| Nanoparticle | α-toxoid (E. coli) |
|---|---|
| formulation | loading rate [%] |
| PVA (1%) | 94 ± 6 |
| TWEEN ® 20 (1%) | 96 ± 1 |
| Sodium cholate (0.05%) | 80 ± 1 |
| SDS (0.01%) | 41 ± 10 |
| CTAB (0.05%) | — |

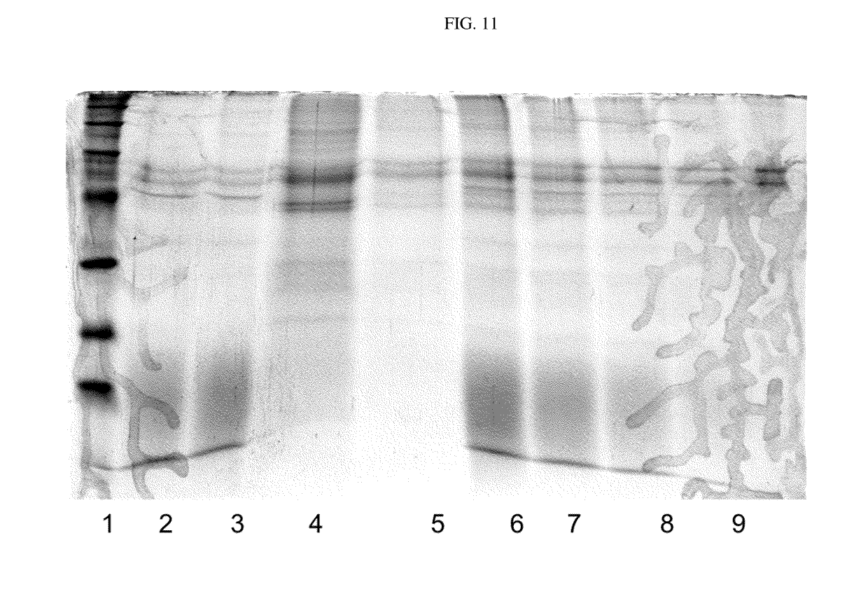

The analysis of the o/w-NP with α-toxoid from Clostridium perfringens was challenging, as the bands of the targeted protein were not clearly visible (FIG. 10). Therefore, in addition to testing the supernatant of the o/w-NP after incubation with the vaccine formulation, the o/w-NP themselves were tested as well. Here, the centrifuged o/w-NP were redispersed with 10% SDS and 2.3% DTT. The test does not give a quantitative result for the loading rate of the α-toxoid on the o/w-NP, because some agglomerates remained after redispersing the sample. A qualitative conclusion could be made nevertheless, as it is clear that at least some part of the α-toxoid from Clostridium perfringens was adsorbed at the surface of the o/w-NP (FIG. 11).

The loading of o/w-NP with the two toxoids could be achieved when using the nonionic surfactants PVA and TWEEN® 20 for the preparation of the nanoparticles. Furthermore, the SDS-NP and sodium cholate-NP could be used for the α-toxoid from E. coli, but not for α-toxoid from Clostridium perfringens. The CTAB-NP are not suitable, as precipitation occurred regardless which of the two toxoids were used. Apparently, the cationic nanoparticles interacted with compounds in the protein solution. The protein solution was manufactured with E. coli or Clostridium perfringens and the purity was under 50%. Recombinant proteins solutions with such a poor purity can contain DNA and proteolytic degradation products, which can lead to aggregates with ionic substances.

The characteristics of the α-toxoid from E. coli are similar to those of BSA and OVA regarding its size. It has a molecular weight of 43 kDa, OVA has a molecular weight of 45 kDa and BSA 66 kDa.

The exact mechanism according to which a protein adsorbs to the surface of PLGA nanoparticles is not completely identified. Hydrophobic interactions of the protein and the o/w-NP seem to play a role in the adsorption of the protein to the PLGA surface. A prediction of the loading rate for a protein cannot be calculated without experimental testing as the mechanism of the adsorption process is not clear. It can be stated that the surfactants that were used to prepare the o/w-NP had an effect on the amount of protein that was loaded to the surface. Different o/w-NP formulations may lead to different results in regard to the loading rate (Table 8). The properties of the protein are also important for the loading rate, as different proteins result in different loading rates when using the same o/w-NP formulation (FIG. 8).

### 3.1.1.6. Investigation of Possible Adsorption Mechanisms

To investigate the protein-nanoparticle interaction several experiments were conducted. The loading rate for different proteins at the surface of nanoparticles with different surfactants has been described above. Moreover, the surface area was investigated regarding its influence on the loading of the proteins. Another aspect, which is crucial for a loading of the proteins on the surface of nanoparticles, is the ability of the protein to accumulate on the surface of the nanoparticle. This means that the protein must be surface-active. Therefore, the interfactial tension of protein solutions was measured to investigate the influence of the surface tension on the loading rate of proteins on the surface of nanoparticles.

The surface tension of the surfactants and the proteins were tested. As expected the surface tension decreases with increasing surfactant and protein concentrations (Table 9 and Table 12). Proteins possess the ability to accumulate at interfaces as they have hydrophilic and lipophilic properties. In comparison, BSA is more surface-active than OVA. The ability of the model proteins to accumulate on interfaces may be one reason, as to why they are adsorbing onto the nanoparticles.

TABLE 9
Surface tension of protein solutions (mean ± SD; n = 3)

Surface tension
[mN/m]

| Concentration protein [mg/ml] | OVA | BSA |
|---|---|---|
| 0.1 | 61.8 ± 0.9 | 58.8 ± 0.2 |
| 0.5 | 57.9 ± 0.3 | 56.2 ± 0.2 |
| 1 | 56.5 ± 3.9 | 53.8 ± 3.2 |

The exact physicochemical interaction between proteins and nanoparticles remains not fully understood. Simple ionic interactions are not the driving force for the nanoparticle-protein complex, as the proteins showed to be adsorbed on anionic surfaces in conditions above their isoelectric point, meaning that the protein itself was also negatively charged. If ionic interactions are responsible for the nanoparticle-protein interaction negatively charged proteins could not adsorb at SDS-NP or sodium cholate-NP. It has been discussed that hydrophobic interactions are responsible for the PLGA-protein complex. A prediction, as to how high an adsorption of a protein is to a given nanoparticle formulation cannot be made, as of now each protein must be tested individually for each nanoparticle formulation.

The fact that proteins adsorb at the surface of nanoparticles must be carefully considered before administration of nanoparticles in-vivo, as proteins in the blood can interact with the nanoparticles.

Also, experiments with nanoparticles in the cell culture must be revisited, when surface modifications are responsible for certain interactions with cells, e.g. uptake in cells. The conditions in-vivo are much more complex and adsorption of proteins onto the surface of nanoparticles can change their properties and thereby change their interactions with cells. Protein free media or even PBS as media should therefore not be used for nanoparticle cell culture experiments as results are most likely not applicable for in-vivo conditions.

### 3.1.1.7. Stability of Nanoparticles

The o/w-NP were tested regarding their stability to determine, if fresh samples must be produced for further experiments or if o/w-NP were sufficiently stable over a prolonged period of time.

The o/w-NP formulations were stored at different temperatures, and either as nanoparticle suspensions or as freeze dried nanoparticles. All samples were stable over 4 weeks regarding their particle size properties when stored at 4° C. as nanoparticle suspensions (Table 10). However, the nanoparticle suspensions were not stable when stored at −20° C. or 20° C. Surprisingly, the nanoparticle suspensions were not completely re-dispersible by simple shaking after storage at −20° C., as aggregates were clearly visible. The nanoparticle suspensions were re-dispersible by ultrasonication for 5 minutes. However, this was not necessary for the original nanoparticle suspension before storage.

Ultrasonication can be harmful to proteins, therefore it should be avoided for specific nanoparticle formulations, as mild processing conditions were a major driver for the development of these nanoparticles. That is why the samples are listed as instable, when stored at −20° C.

Freeze dried nanoparticles were stable over 4 weeks of storage at 4° C. and easily re-dispersible, except for the CTAB-NP. Trehalose was added to the nanoparticle suspension at a concentration of 5% prior freeze drying as a cryoprotectant.

TABLE 10
Stability of o/w-nanoparticles over 4 weeks storage; Samples were stored
either as nanoparticle suspension at −20° C., 4° C. or 20° C.
or as freeze dried nanoparticles at 4° C. (—) indicates that sample
was not redispersible or aggregation occurred

| | Nanoparticle | | Freeze dried | |
|---|---|---|---|---|
| | suspension | | nanoparticles | |
| Nanoparticle formulation | −20° C. | 4° C. | 20° C. | 4° C. |
| PVA-NP | — | 146.9 nm | — | 139.3 nm |
| TWEEN ® 20-NP | — | 183.2 nm | — | 173.3 nm |
| Sodium-cholate-NP | — | 126.7 nm | — | 164.6 nm |
| SDS-NP | — | 211.9 nm | — | 187.4 nm |
| CTAB-NP | — | 220.7 nm | — | — |

### 3.1.1.8. Characterization of Lipopolysaccharides Loaded Nanoparticles

The findings about the nanoparticles with the surface adsorbed protein with different surfactants showed a simple method to prepare protein loaded, polymeric nanoparticles. Derived from this method, LPS-NP were prepared, by using the emulsification-evaporation method. LPS-NP were developed to be used as an adjuvant formulation to be applied in combination with an antigen. Here, LPS was added to the outer water phase during the emulsification-evaporation method, technically acting as the surfactant, leading to polymeric nanoparticles with surface adsorbed LPS. LPS-NP are currently investigated regarding its potential as vaccine adjuvants. In this work, a novel preparation of the LPS-NP was employed.

LPS-NP were prepared by a simple emulsification-evaporation method and the active ingredient LPS also operated as the surfactant so that no additional surfactant was necessary. The resulting LPS-NP had a LPS concentration of 1 mg/ml and a PLGA concentration of 2.5 mg/ml. As the quantitative measurement of LPS is normally very complex an indirect method was applied to quantify the amount of LPS on the nanoparticles. FITC-labeled LPS was used to prepare LPS-NP, afterwards the nanoparticles were centrifuged and the supernatant was tested regarding its FITC-LPS content. It was observed that almost 70% of the LPS was bound to the nanoparticles (Table 11). The prepared LPS-NP had a particle size with a mean diameter of around 200 nm. This particle size was desired as LPS-NP in that size range showed an improved immune response in mice in previous studies. The L2 formulation was subsequently used as an adjuvant formulation in combination with CpG in the mice studies, as LPS and CpG are PAMPs and therefore TLR-4 receptor agonist that induce dendritic cell maturation and T-lymphocytes activation.

TABLE 11
Particle size and loading rate of LPS-NP (mean ± SD; n = 3)

| Sample | LPS formulation | Mean diameter [nm] | Loading rate [%] |
|---|---|---|---|
| L1 | 2 ml of LPS-solution | 227 ± 13 | — |
| | [1 mg/ml] | | |
| L2 | 4 ml of LPS-solution | 175 ± 8 | — |
| | [1 mg/ml] | | |
| L3 | 3.8 ml of LPS- | 179 ± 2 | 68 ± 2 |
| | solution [1 mg/ml] + | | |
| | 0.2 ml FITC-LPS | | |

### 3.1.2. In Vivo Studies

The ability of proteins to adsorb at the o/w-NP surface was demonstrated in this work. The surface of the nanoparticles was modified, depending on the surfactant that was used. The different formulations were characterized, amongst other things, in regard to the loading rate of the proteins and the ability to be taken up by cells. LPS-NP already showed the potential to increase an immune response, therefore the LPS-NP prepared in this work with an emulsification-evaporation method were tested regarding their ability to influence the immune response.

To investigate the adjuvant effect of the o/w-formulations in-vivo studies were conducted. IgG titers were examined to quantify the immune response after immunization. OVA was used as a model antigen, as it already proved to be a suitable antigen in mice for immunization studies and the IgG concentration was tested using an ELISA-Kit specifically for Anti-OVA IgG. Blood samples were drawn on study day 0, study day 20 and study day 35. The first blood sample was simply to control, if the antibody titer was not elevated before the start of the immunization. On day 20 a low IgG antibody titer was expected, but conclusions about the adaptive immune response cannot be made, as the immune effects are mainly results of the innate immune system. Essential for an evaluation of the adaptive immune response are the IgG-titers at study day 35. After the second vaccination IgG is being released by B-cells, and the concentration reduces very slowly over a prolonged period of time.

### 3.1.2.1. In Vivo Testing of Different Adjuvants

In the in-vivo study different adjuvant formulations were tested. CFA/IFA is a well-known adjuvant that elicits very high antibody titers, but is not used in humans or farm animals due to toxicity issues. As the use of this adjuvant is very dangerous, the mice were anaesthetized with isofluran before immunization. CFA/IFA was used as a positive control group. The test groups were LPS-NP with CpG and LPS in solution with CpG. PBS with OVA was used as another control group to see if the test groups show a beneficial influence regarding the immune response in mice following their administration.

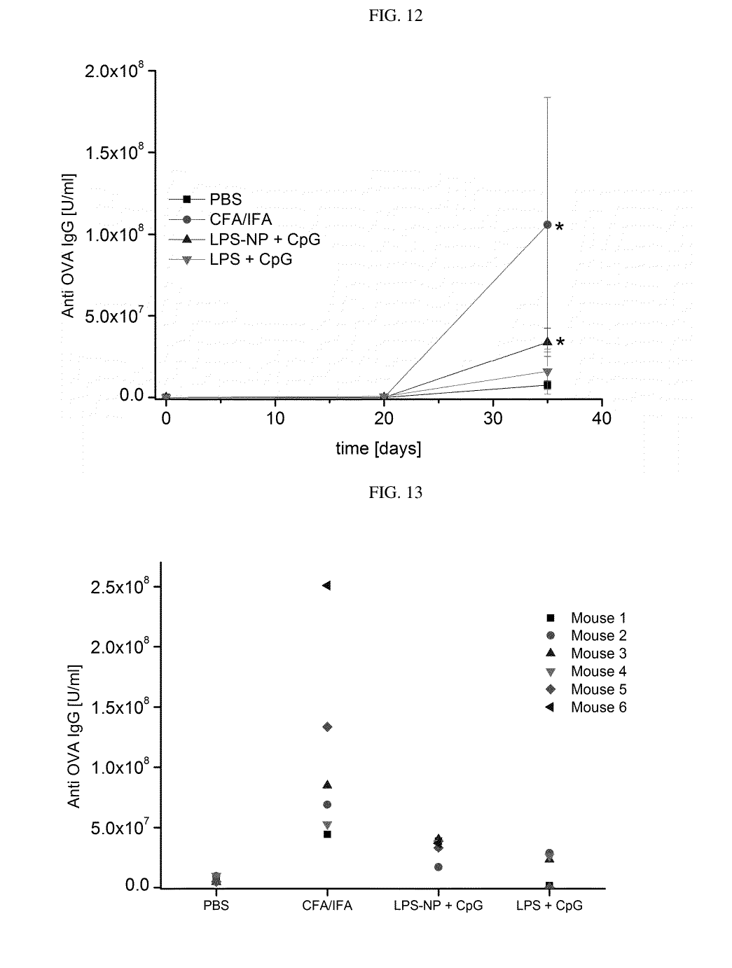

The diagram of the IgG antibody response shows the typical process of an immunization study (FIG. 12). The IgG titers are at 0 U/ml before the first immunization for all formulations, meaning that the mice did not possess any anti-OVA IgG before the study. Before the second immunization at SD 20, the IgG titers are not higher than 5×10⁶ for any group. This is also typical for immunization studies, where the immunizations are three weeks apart. Following the first immunization IgG starts to be build up after two weeks, but a fast decrease of IgG occurs afterwards. After the second immunization, a high IgG concentration is immediately existent that decreases very slowly over a long period of time, depending on the intensity of the immune response.

The CFA/IFA formulations showed the highest immune response (FIG. 12). The difference to the other groups was significant. In addition, the LPS-NP group was significantly better than the LPS and PBS group regarding its antibody response. FIG. 13 shows the Serum IgG-titers of each mouse after study day 35.

Here, it was shown that the LPS-NP caused a significantly higher antibody response than the LPS solution, meaning that the TLR-4 agonist LPS is taken up by dendritic cells and macrophages to provoke a strong immune response. LPS is one class of PAMP that leads to the maturation of dendritic cells and subsequently to the activation of T-lymphocytes.

It can be concluded that LPS-NP are favorable compared to LPS in solution. The immune response in the LPS-NP and LPS in solution group is of course also a consequence of the CpG, but since it was present in both groups, the difference between the groups can be attributed to the usage of nanoparticles. The nanoparticles are able to get inside the cells, TLR are located at the cell surface as well as inside cells at endosomes. The application of LPS-NP is advantageous as LPS does not only fulfill its agonistic properties on TLR on the cell surface, but also inside the cells on endosomes following a better uptake into the cells with the nanoparticles.

### 3.1.2.2. Lipopolysaccharides Loaded Nanoparticles and Ovalbumin Loaded Nanoparticles in Mice

The next in-vivo study was performed to test the different OVA loaded o/w-formulations regarding their ability to enhance the immune response compared to LPS-NP with OVA. As OVA is located onto the surface of the o/w-NP, it is fast released and can be recognized immediately by cells of the immune system, like dendritic cells and macrophages. Due to its adsorption on nanoparticles, OVA might even be better taken up by cells compared to OVA alone.

It must be noted that the loading rate of the different o/w-NP formulations were different. Since no washing step was performed, all formulations had the same OVA amount within one nanoparticle suspension, but the ratio of OVA loaded on the o/w-NP and OVA in solution was different, e.g. for the CTAB-NP no loading was observed.

All o/w-NP formulations that were injected also contained LPS-NP and CpG in the same concentration as in the control group.

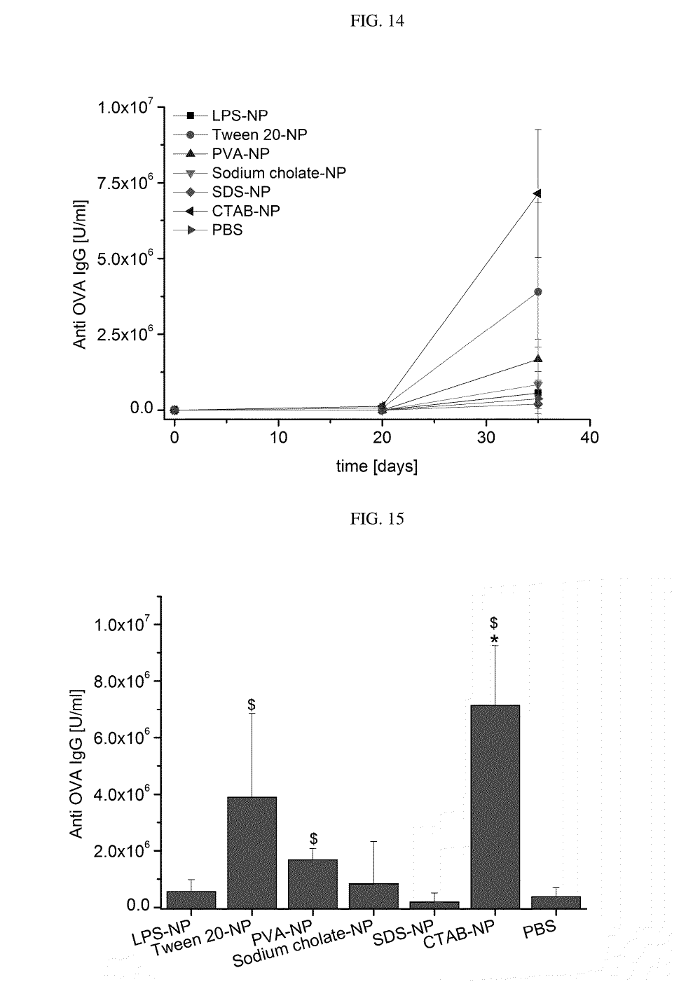

As already shown in the previous in-vivo studies, the time-diagram of the antibody response had a typical progress (FIG. 14).

The CTAB-NP showed the highest immune response following administration in mice (FIG. 15). The antibody response was significantly higher compared to all other groups. The OVA loaded TWEEN® 20-NP and OVA loaded PVA-NP elicited a significantly higher immune response compared to the control group PBS without additional LPS, but the difference compared to the control group with LPS-NP was not significant. The anionic o/w-NP formulations did not provoke a higher immune response than the PBS control group.

For the SDS-NP it can be assumed that the SDS linearizes the protein, therefore reducing its immunogenic properties. It was expected that significantly higher immune response would be produced by the other formulations, because of the good cell uptake of the PVA-NP, TWEEN® 20-NP and sodium cholate-NP into macrophages coupled with high loading rates for these o/w-NP formulations. The loading of the nonionic o/w-NP was relatively high (71%-83%). In cell culture studies it was already shown that nonionic o/w-NP can be taken up by cells and that they are located inside the cells. However, a significant effect regarding their antibody response in mice was not observed. It can be hypothesized that the cell uptake and delivery of the protein into the cells of the immune system is not the mode of action here, but that the toxic properties of the nanoparticles play an important role.

The high immune response of the CTAB-NP might be explained by their toxic characteristics. As previously described, the CTAB-NP and TWEEN®-NP showed the highest toxicity in the cell culture model using RAW 264.7 cells. Those two formulations showed the highest antibody response after immunization in mice. The TWEEN®-NP showed a good loading with OVA, but the CTAB-NP were not loaded at all with OVA. Therefore, the effect of an enhanced cell uptake via nanoparticles of OVA cannot be the reason for the high immune response. More likely is that, because of its toxic potential, cells of the immune system are being released to the injection site, making it more probable for OVA to be processed. This is a mode of action similar to other adjuvants, where the cell uptake is not the primary concern. Many adjuvants induce an inflammation, e.g. CFA/IFA and Alum, thereby activating the innate immune system and subsequently leading to adaptive immunity in combination with an immunogenic. Therefore, the activation of the innate immune system by the nanoparticles might be responsible for the higher immune response compared to the control group.

### 3.1.2.3. Influence of Nanoparticle Concentration on Immune Response in Mice

In the previous in-vivo study it was observed that CTAB-NP had a significant influence on the immune response after immunization in mice in combination with LPS-NP and OVA. The nonionic o/w-NP formulations also elicited high immune responses, but a statistical difference was not obtained. The anionic o/w-formulations showed the smallest antibody response; therefore further testing was conducted without these anionic o/w-NP formulations.

Here, the influence of the nanoparticle concentration was determined. As in the previous study, all formulations contained LPS-NP and CpG in the same concentration as in the control group. The concentration of the PLGA-NP was varied, but the surfactant content stayed the same for each o/w-NP formulation. A difference in the immune response could therefore be attributed to the nanoparticles and not to the “free” surfactant in solution.

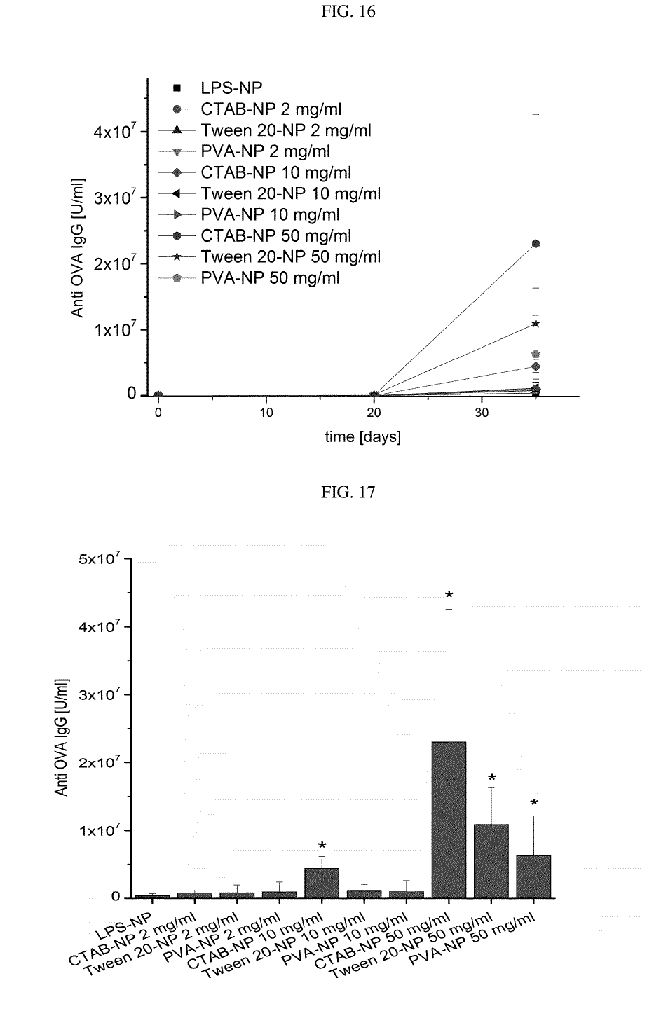

As already shown in the previous in-vivo studies, the time-diagram of the antibody response had a typical progress (FIG. 16).

When comparing the IgG titers at SD 35, it can be clearly seen that the dose of the nanoparticles has an effect on the immune response (FIG. 17).

Groups II-IV with a nanoparticle concentration of 2 mg/ml for each o/w-NP formulation have the smallest IgG titers, followed by the formulations with 10 mg/ml, while formulations with 50 mg/ml nanoparticles provoked the highest immune response.

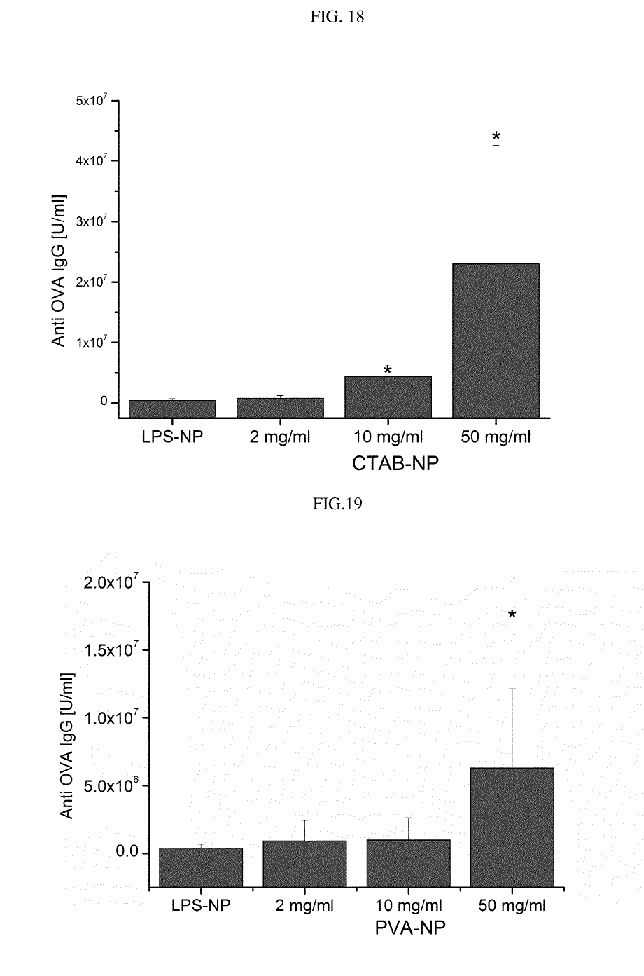

When comparing the CTAB-NP at different concentrations with each other it can be observed that, when using the highest nanoparticle concentration (50 mg/ml), a statistical difference to the other used concentrations (10 mg/ml and 2 mg/ml) and to the control group exists (FIG. 18). As already seen in the previous study, the CTAB-NP formulation with 10 mg/ml elicits a higher antibody response than the control group. The results of the previous study could be confirmed in that regard.

These experiments demonstrate that the concentration of the nanoparticles has an influence on the antibody response in mice. As discussed earlier, an inflammatory effect might be the cause for the immune response following immunization with CTAB-NP. Therefore, a higher immune response with a higher nanoparticle concentration is comprehensible.

The inflammatory effect of CTAB-NP was visible at a concentration of 50 mg/ml as four out of five mice had a mild inflammation at the injection site. Two weeks after the immunization in the neck fold the immunization was still visible on the neck. This suggests that a strong inflammation occurred during immunization, making the CTAB-NP at such high concentrations inapplicable as adjuvants, as safety and tolerability is of utmost importance for adjuvants. Adjuvants that on the one hand elicit a very strong antibody response and on the other hand are toxic or highly inflammatory are not suitable for parental application. One example for this would be CFA/IFA, where high antibody titers can be obtained when a vaccine is administered with CFA/IFA, but due to toxicity issues CFA/IFA is only used for research and development.

The PVA-NP showed a similar behavior as the other o/w-NP formulations, as the IgG titers at SD 35 increased with increasing concentrations of the nanoparticles (FIG. 19). The PVA-NP with the highest nanoparticle concentration (50 mg/ml) provoked an antibody response at SD 35 that was significantly higher compared to the control group. The titers caused by PVA-NP at the other concentrations were also higher than the control group, but a statistically significant difference was not observed. The results of the previous study could be confirmed in that regard.

An inflammation at the injection site was not visible for any concentration of PVA-NP that was used for this immunization study, making the PVA-NP better tolerable to the CTAB-NP at the highest concentration, even though it must me noted that the CTAB-NP at 50 mg/ml provoked a much higher immune response than the PVA-NP.

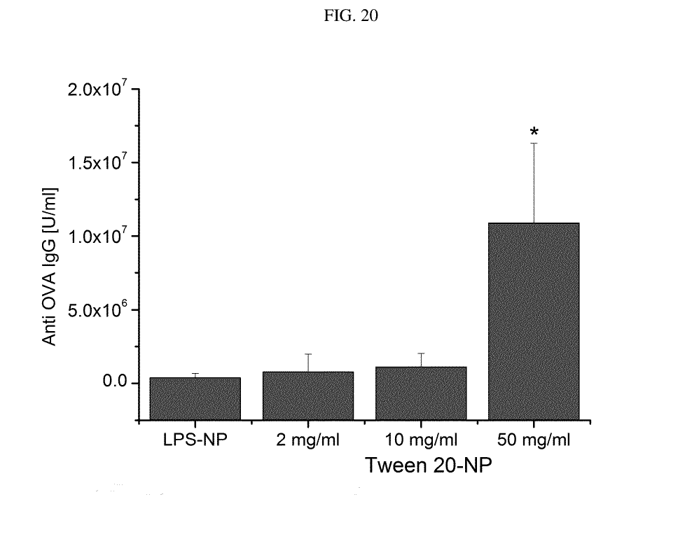

The TWEEN® 20-NP showed a similar behavior as the other o/w-NP formulations, as the IgG titers at SD 35 increased with increasing concentrations of the nanoparticles (FIG. 20). TWEEN®-NP at a concentration of 50 mg/ml provoked an immune response that was significantly higher than the LPS-NP (control group). At the other concentrations of the TWEEN® 20-NP a significant difference compared to the control group was not observed, even though a slight increase of the immune response was visible when comparing the TWEEN® 20-NP with the control group. The results of the previous study could be confirmed in that regard.

TWEEN® 20-NP like the also non-ionic PVA-NP did not induce any visible inflammation at the injections site, meaning that they are superior to the CTAB-NP regarding their safety and tolerability.

The in-vivo studies revealed insight into the strength of immune responses following the application of different adjuvants.

The testing of different adjuvant formulations revealed that LPS-NP in combination with CpGare beneficial to LPS in solution in combination with CpG. This is of particular interest as TLR-4 agonists pose as an intriguing way to trigger a strong immune response to obtain adaptive immunity. LPS itself is too toxic to be used in humans, but similar TLR-4 agonists are already being tested in clinical trials. Preparation of TLR-4 agonists with nanoparticles might be beneficial in terms of the adjuvant effect as the LPS-NP showed an improved immune response compared to the LPS in solution. Furthermore, it was seen that CFA/IFA showed significantly higher antibody responses than the LPS-NP, but the CFA/IFA formulation was just tested to have a positive control. Due to toxicity issues CFA/IFA is obsolete, although very high antibody responses can be produced with CFA/IFA.

The experiments with the five different o/w-NP formulations revealed that the CTAB-NP elicit the highest antibody response. This was particularly surprising as it was previously observed that no OVA was adsorbed at the surface of the CTAB-NP, meaning that the uptake into cells with the nanoparticles was not the mode of action here. Besides, CTAB-NP showed to have the lowest uptake rate into cells in cell culture experiments conducted with RAW 264.7 cells. Rather than a drug targeting with CTAB-NP, an activation of the immune system due to inflammation seems to be responsible for the high immune response. This hypothesis is supported by the fact that the antibody response increases with higher CTAB-NP concentration and inflammation was visible at the injection site for CTAB-NP with the highest nanoparticle concentration.

The antibody response for the nonionic o/w-NP formulations was also slightly increased compared to the control group. The nonionic o/w-NP formulations did not alter the immune response compared to the control at a concentration of the nanoparticles of 10 mg/ml. A significant difference in the antibody response for the nonionic o/w-NP formulations was just observed at the highest nanoparticle concentration, suggesting that a dose-dependent inflammatory effect may also be the mode of action here. Even for low dosage nanoparticle formulations a high loading rate, similar to high dosage nanoparticle formulations, was observed, which leads to the assumption that not the loading rate, but the nanoparticle dosage is deciding for the immune response.

It has been reported that nanoparticles and microparticles with conjugated antigens show a stronger immune response than soluble antigen. In these studies a size dependent effect was seen, as particles smaller than 10 μm showed a significantly higher immune response. Nanoparticles in a size range of 50-200 nm elicited a stronger immune response than larger and smaller particles. It has also been previously investigated that dendritic cells are able to internalize PLGA-NP.

However, nanoparticles used in those studies were prepared by a double-emulsion method and it was postulated that the antigen is encapsulated inside the nanoparticle. In this work we showed that the protein is not located inside the nanoparticles, but adsorbed at the particle surface. This does not contest the findings of the other studies, but the mode of action must be revisited, as phagocytosis of the nanoparticles with the encapsulated antigen was considered crucial for the stimulation of the immune system.

LPS loaded nanoparticles combined with antigen showed a proinflammatory effect, which ultimately led to an increase of the immune response in mice studies (Demento et al., 2009). In the study of Demento et al. it was also hypothesized that the antigen encapsulated in a nanoparticulate carrier was better internalized into the dendritic cells.

However, the strongest immune response in this work was observed for CTAB-NP, which did not have OVA adsorbed at the particle surface. OVA was dissolved in the nanoparticle suspension, suggesting that an uptake into the cells with the nanoparticle was not important for the strong antibody response. Moreover, it can be hypothesized that the CTAB-NP activate the immune system due to their toxic surface properties.

The potential of nanoparticles as vaccine adjuvants has already been tested in-vivo and proven successful in regard to a significantly higher immune response compared to soluble antigen (Demento et al., 2009). However, a comprehensive understanding about the mode of action of nanoparticles as vaccine adjuvants is still not available.

Further testing would be required to investigate the exact mode of action of the o/w-NP, prepared in this work. The nonionic o/w-NP were only an improvement at a nanoparticle concentration of 50 mg/ml, regarding their adjuvant capabilities. The CTAB-NP were showing an adjuvant effect at lower concentrations, but those nanoparticles are most likely not suitable for vaccinations, due to their toxic potential. An inflammation was visible at the injection site in the mice studies conducted in this work, when using CTAB-NP.

PLGA nanoparticles already showed to increase the immune response as vaccine adjuvants in mice. However, further studies are necessary, to determine if the nanocarriers are an improvement to alternative adjuvant systems as PLGA-NP are very expensive and the toxic effects following administration might outweigh the benefits.

### 4. Summary and Conclusion

Live, attenuated vaccines consist of virus or bacteria that are not virulent, but have the ability to proliferate. Therefore, a strong immune response can be generated, as the vaccine is distributed throughout the body, due to the proliferation properties of the microorganisms. Mutation of the vaccine can cause the microorganisms to reverse to virulence. Therefore, inactivated, non-live vaccines are more desirable on the grounds of safety issues. However, inactivated vaccines rarely induce a strong enough immune response to achieve adaptive immunity. That is why adjuvants are essential for successful vaccinations.

The objective of this work was to develop protein loaded and nanoparticles as parental carrier systems for the antigen delivery, as particulate, polymeric carrier systems have a huge potential to pose as adjuvants.

Nanoparticles have a similar size as pathogens, making them particularly interesting for antigen delivery. Besides, nanoscale drug delivery systems can enhance the transport of the active compound across absorption barriers, leading to high amounts of antigen within dendritic cells and macrophages.

A simplified method was used to obtain protein loaded nanoparticles. The particles were prepared using an emulsification-evaporation method followed by an incubation process, in which the hydrophilic drug, in this case OVA or BSA, was adsorbed at the nanoparticle surface. The obtained PLGA-NP were prepared with different surfactants, yielding in nanoparticles with modified surface properties. The nonionic surfactants TWEEN® 20 and PVA, the anionic surfactants SDS and sodium cholate and the cationic surfactant CTAB were utilized. The particles were characterized regarding their particle size, loading rate with OVA and BSA, as well as their release profile. A fast release within one hour was observed for all formulations. In most cases BSA was adsorbed at a higher rate than OVA on the nanoparticles, an exact mode of action on how the adsorption takes place is still unknown.

The five different nanoparticle formulations were also tested for their potential to adsorb Clostridium perfringens α-toxoids. Two different α-toxoids were tested. One was formalin inactivated α-toxoid from Clostridium perfringens and the other was expressed in genetically modified E. coli. Using SDS-PAGE it could be observed that both proteins were not pure, leading to aggregation with the cationic CTAB-NP. The anionic SDS-NP and sodium-cholate-NP were incompatible with the α-toxoid from Clostridium perfringens. PVA-NP and TWEEN® 20-NP showed a satisfying behavior in regard to the compatibility with the proteins. Furthermore, it was discovered that most of the α-toxoid was adsorbed at the surface of the nonionic nanoparticles.

The exact mechanism in which the protein adsorbs to nanoparticles is the target of many studies, but it has not been fully explained. It is believed that the proteins arrange themselves as a “protein corona” around the nanoparticles. Hydrophobic interactions seem to be the deciding force during the adsorption of proteins on nanoparticles.

In immunization studies with BALB/c mice the immune response following application of the different OVA loaded nanoparticle formulations was determined. CTAB-NP showed a significantly higher antibody response compared to the other formulations. This was very surprising as OVA was not located at the surface of the CTAB-NP, but was just dissolved in the nanoparticle suspension. An increased transport into the cells with the CTAB-NP was therefore not likely. The second highest antibody response was elicited by TWEEN® 20-NP, which suggests that the toxicity of these two formulations might be the reason for the high antibody response. When investigating the influence of different concentrations of the nanoparticle formulations, it was revealed that at the highest CTAB-NP concentration a severe inflammation at the injection site was visible. This suggests that a pronounced inflammation occurred, leading to an accumulation of cells of the immune system like dendritic cells and macrophages, which are crucial for adaptive immunity.

Another adjuvant formulation that was developed were LPS-NP. LPS are TLR-4 agonists and therefore an interesting substance as they activate and facilitate the maturation of dendritic cells. An emulsification-evaporation method was used for the preparation of LPS-NP. It was determined that almost 70% of the LPS was bound on the nanoparticles. Using an in-vivo study the adjuvant potential of LPS-NP was investigated. It can be concluded that LPS-NP, when combined with CpG, was significantly beneficial regarding its antibody response compared to LPS and CpG in solution. These findings are interesting for the development of novel adjuvants, as new TLR-ligands can be used in combination with nanoparticles to improve their adjuvant properties.

TABLE 12
List of substances used in experiments

| Substance | Manufacturer |
|---|---|
| Bovine Serum Albumin | Boehringer Ingelheim |
| CpG | Invivogen |
| CTAB | Roth |
| Disodium phosphate | Sigma Aldrich |
| DTT | Roth |
| Ethyl acetate | ZVE |
| FBS | Sigma Aldrich |
| Freund's Adjuvant, complete | Sigma Aldrich |
| Freund's Adjuvant, incomplete | Sigma Aldrich |
| LPS | Sigma Aldrich |
| Methylene chloride | ZVE |
| MTT | Sigma Aldrich |
| Nile red | Sigma Aldrich |
| Ovalbumin | Sigma Aldrich |
| Penicillin-Streptomycin | Sigma Aldrich |
| PLGA | Evonik |
| Potassium chloride | Sigma Aldrich |
| Potassium dihydrogen phosphate | Sigma Aldrich |
| PVA | Kuraray |
| SDS | Sigma Aldrich |
| Sodium chloride | Sigma Aldrich |
| Sodium cholate | Sigma Aldrich |
| TWEEN ® 20 | Roth |
| TWEEN ® 80 | Roth |
| α-toxoid | Boehringer Ingelheim |

TABLE 13
List of Abbreviations

| | |
|---|---|
| ° C. | degree Celsius |
| API | active pharmaceutical ingredient |
| BCA | bicinchoninic acid |
| CFA | Complete Freund's Adjuvant |
| CLSM | confocal laser scanning microscopy |
| CTAB | cetyltrimethylammonium bromide |
| Da/kDa | Dalton/kilodalton |
| DMEM | Dulbecco's Modified Eagle Medium |
| DMSO | dimethyl sulfoxide |
| DTT | dithiothreitol |
| EE | encapsulation efficiency |
| ELISA | enzyme-linked immunosorbent assay |
| FE-SEM | field emission-scanning electron microscope |
| FITC | fluorescein isothiocyanate |
| g | acceleration of gravity |
| G | Gauge |
| IFA | Incomplete Freund's Adjuvant |
| IgG | immunoglobulin G |
| kV | kilovolt |
| LPS | lipopolysaccharides |
| MTT | methylthiazolyldiphenyltetrazolium bromide |
| NP | nanoparticles |
| o/w | oil-in-water |
| OVA | ovalbumin |
| PAGE | polyacrylamide gel electrophoresis |
| PAMP | pathogen associated molecular pattern |
| PCS | photon correlation spectroscopy |
| PDI | polydispersity index |
| PLGA | polylactid-co-glycolid |
| PVA | polyvinyl alcohol |
| rcf | relative centrifugal force |
| rpm | revolutions per minute |
| s.c. | subcutaneous |
| SD | standard deviation |
| SDS | sodium dodecyl sulfate |
| SEM | scanning electron microscope |
| STIKO | german: Ständige Impfkommission des Robert |
| | Koch-Instituts |
| TLR | Toll-like receptor |
| w/o | water-in-oil |
| w/o/w | water-in-oil-in-water |

### REFERENCES

Demento, S. L., Eisenbarth, S. C., Foellmer, H. G., Platt, C., Caplan, M. J., Mark Saltzman, W., Mellman, I., Ledizet, M., Fikrig, E., Flavell, R. A., Fahmy, T. M., 2009. Inflammasome-activating nanoparticles as modular systems for optimizing vaccine efficacy. Vaccine 27, 3013-3021.
Gutierro, I., Hernandez, R. M., Igartua, M., Gascon, A. R., Pedraz, J. L., 2002a. Influence of dose and immunization route on the serum Ig G antibody response to BSA loaded PLGA microspheres. Vaccine 20, 2181-2190.
Vauthier, C., Bouchemal, K., 2009. Methods for the Preparation and Manufacture of Polymeric Nanoparticles. Pharm Res 26, 1025-1058.
Wachsmann, P., Lamprecht, A., 2012. Polymeric nanoparticles for the selective therapy of inflammatory bowel disease. Methods Enzymol 508, 377-397.
Waeckerle-Men, Y., Groettrup, M., 2005. PLGA microspheres for improved antigen delivery to dendritic cells as cellular vaccines. Adv Drug Deliv Rev 57, 475-482.

1. Amphiphile coated nanoparticle, wherein said nanoparticle is composed of:
a. a solid core consisting of a biodegradable polymer, wherein optionally solvent molecules are included in the interior of the solid core;
b. an amphiphile shell disposed over said solid core; and
c. optionally, one or more antigens attached to said amphiphile and/or said solid core.

2. The amphiphile coated nanoparticle of claim 1 having a diameter lower than 250 nm or preferably having a size within a range of from 50 to 200 nm.

3. The amphiphile coated nanoparticle according to claim 1, wherein said biodegradable polymer is a synthetic polymer, and wherein said synthetic polymer is preferably selected from the group consisting of polylactides, polyglycolides, polylactic polyglycolic copolymers, polyesters, polyethers, polyanhydrides, polyalkylcyanoacrylates, polyacrylamides, poly(orthoters), polyphosphazenes, polyamino acids, and biodegradable polyurethanes.

4. The amphiphile coated nanoparticle according to claim 1, wherein said biodegradable polymer is selected from the group consisting of poly(lactic-co-glycolic acid) (PLGA), Poly(Lactide-co-Glycolide) (PGA), Poly(lactic acid) (PLA), poly(ε-Caprolactone) PCL, Poly(methyl vinyl ether-co-maleic anhydride), PEG-PCL-PEG, and Polyorthoesters.

5. The amphiphile coated nanoparticle according to claim 1, wherein said amphiphile is a surfactant or a TLR (Toll like receptor) agonist.

6. The amphiphile coated nanoparticle according to claim 5, wherein said amphiphile is a surfactant selected from the group consisting of non-ionic, anionic, and cationic surfactants, and wherein said amphiphile is preferably:
a. a non-ionic surfactant selected from the group consisting of polyoxyethylene sorbitan fatty acid esters, sorbitan fatty acid esters, fatty alcohols, alkyl aryl polyether sulfonates, and dioctyl ester of sodium sulfonsuccinic acid;
b. an anionic surfactant selected from the group consisting of sodium dodecyl sulfate, sodium and potassium salts of fatty acids, polyoxyl stearate, polyyoxylethylene lauryl ether, sorbitan sesquioleate, triethanolamine, fatty acids, and glycerol esters of fatty acids; and
c. a cationic surfactant selected from the group consisting of didodecyldimethyl ammonium bromide, cetyl trimethyl ammonium bromide, benzalkonium chloride, hexadecyl trimethyl ammonium chloride, dimethyidodecylaminopropane, and N-cetyl-N-ethyl morpholinium ethosulfate.

7. The amphiphile coated nanoparticle according to claim 1, wherein said amphiphile is a surfactant selected from group consisting of Polyvinyl alcohol (PVA), Polysorbate 20 (TWEEN® 20), Sodium dodecyl sulfate (SDS), Sodium cholate, and Cetyltrimethylammonium bromide (CTAB).

8. The amphiphile coated nanoparticle according to claim 1, wherein said amphiphile and/or said one or more antigens is selected from the group consisting of TLR (Toll like receptor) agonists, and wherein said amphiphile and/or said one or more antigens is preferably selected from the group consisting of a TLR1 agonist, a TLR2 agonist, and a TLR4 agonist.

9. The amphiphile coated nanoparticle according to claim 8, wherein said TLR agonist is selected from the group consisting of: lipopolysaccharide (LPS) or a derivative thereof, lipoteichoic acid (LTA), Pam(3)CysSK(4) ((S)-[2,3-5w(palmitoyloxy)-(2-RS)-propyl]-N-palmitoyl-(R)-Cys-(S)-Ser-(S)-Lys₄-OH or Pam₃-Cys-Ser-(Lys)), Pam3Cys (S-[2,3-bis(palmitoyloxy)-(2RS)-propyl]-N-palmitoyl-(R)-cysteine or tripalmitoyl-S-glyceryl cysteine), Cadi-05, ODN 1585, zymosan, synthetic triacylated and diacylated lipopeptides, MALP-2, tripalmitoylated lipopeptides, a compound having a 2-aminopyridine fused to a five membered nitrogen-containing heterocyclic ring, Polyriboinosinic-polyribocytidylic acid (poly IC), a CpG oligodeoxynucleotides (ODNs), monophosphoryl lipid A (“MPL”), an imidazoquinoline compound (e.g. an amide substituted imidazoquinoline amine), a benzimidazole derivative, a C8-substituted guanine ribonucleotide, an N7, C8-substituted guanine ribonucleotide, bacteria heat shock protein-60 (Hsp60), peptidoglycans, flagellins, mannuronic acid polymers, flavolipins, teichuronic acids, ssRNA (single stranded RNA), dsRNA (double stranded RNA), or a combination thereof.

10. The amphiphile coated nanoparticle according to claim 1, wherein said amphiphile is a TLR agonist selected from the group consisting of LPS or a derivative thereof, and LTA and/or wherein said one or more antigens is selected from the group consisting of proteins and peptides, and wherein the one or more antigen is preferably an alpha-toxin, more preferably Clostridium perfringens α-toxin or α-toxoid.

11. The amphiphile coated nanoparticle according to claim 1, wherein said amphiphile and/or said one or more antigens is a TLR4 agonist selected from LPS or a derivative thereof, and wherein said derivative of LPS is preferably selected from the group consisting of monophosphoryl lipid A (MPL), 3-O-deacylated monophosphoryl lipid A (3D-MPL), and Glucopyranosyl Lipid A (GLA).

12. The amphiphile coated nanoparticle according to claim 11, wherein said Glucopyranosyl Lipid A (GLA) is a compound of formula (I): 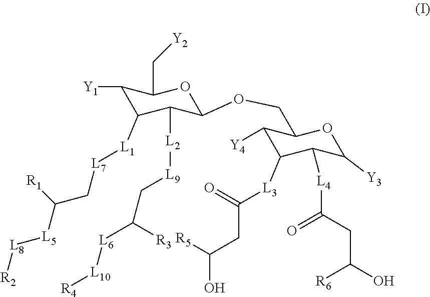
or a pharmaceutically acceptable salt thereof, wherein:
L₁, L₂, L₃, L₄, L₅ and L₆ are the same or different and are independently selected from —O—, —NH— and —(CH₂)—;
L₇, L₈, L₉ and L₁₀ are the same or different and are each independently either absent or —C(═O)—;
Y₁ is an acid functional group and is preferably —OP(═O)(OH)₂;
Y₂ and Y₃ are the same or different and are each independently selected from —OH, —SH, and an acid functional group;
Y₄ is —OH or —SH;
R₁, R₃, R₅ and R₆ are the same or different and are independently C₈₋₂₀ alkyl; and
R₂ and R₄ are the same or different and are independently C₆₋₂₀ alkyl,
and wherein
Y₂, Y₃ and Y₄ are preferably each —OH; and/or
R₁, R₃, R₅ and R₆ are the same or different and, preferably, are independently C₈₋₁₃ alkyl; and/or
R₂ and R₄ are the same or different and, preferably, are independently C₆₋₁₁ alkyl.

13. A method for the production of the amphiphile coated nanoparticle according to claim 1 comprising or consisting of the steps of:
a. adding (i) an organic solvent containing the biodegradable polymer to (ii) an aqueous phase containing the amphiphile;
b. sonicating the combined organic solvent and aqueous phase at an energy sufficient to form a stable emulsion;
c. evaporating the organic solvent from the stable emulsion;
d. optionally, separating the resulting nanoparticles from at least part of the remaining aqueous phase and preferably freeze drying the resulting nanoparticles and/or storing the resulting nanoparticles at a temperature of not more than 7° C.; and
e. optionally, adding the one or more antigens or a composition comprising the one or more antigens to the remaining aqueous phase and/or the resulting nanoparticles.

14. The method of claim 14, wherein said organic solvent is a nonpolar organic solvent, and wherein said nonpolar organic solvent is preferably selected from the group consisting of ethyl acetate, methylene chloride, chloroform, tetrahydrofuran, hexafluoroisopropanol, and hexafluoroactone sesquihydrate.

15. The amphiphile coated nanoparticle according to claim 1, for use as an immunomodulatory agent, in particular as an adjuvant, or for use in a method for stimulating an immune response in a subject.

16. Use of the amphiphile coated nanoparticle according to claim 1, as an adjuvant for the manufacture of a vaccine, wherein the vaccine preferably comprises an antigen.

17. A method for stimulating an immune response in a subject comprising administering a composition comprising one or a plurality of the nanoparticle according to claim 1 to said subject.
